(19) United States
(12) Patent Application Publication
(10) Pub. No.: US 2023/0364215 A1
(43) Pub. Date: Nov. 16, 2023

(54) COMPOSITIONS COMPRISING INACTIVATED MICROBES, AND METHODS FOR USE AND PRODUCTION THEREOF

(71) Applicant: Colorado State University Research Foundation, Fort Collins, CO (US)

(72) Inventors: Ramond P. Goodrich, Fort Collins, CO (US); Richard Bowen, Fort Collins, CO (US); Marcela Henao-Tamayo, Fort Collins, CO (US)

(21) Appl. No.: 18/029,640

(22) PCT Filed: Oct. 1, 2021
(86) PCT No.: PCT/US21/53160

§ 371 Date: Mar. 30, 2023

Related U.S. Application Data
(60) Provisional application No. 63/086,792, filed on Oct. 2, 2020.
(60) Provisional application No. 63/241,902, filed on Sep. 8, 2021.

(51) Int. Cl. A61K 39/04; A61K 41/17; A61P 31/06; A61K 35/74

(52) U.S. Cl. CPC A61K 39/04; A61K 41/17; A61P 31/06; A61K 35/74; A61K 2039/521

(57) ABSTRACT

Provided herein are methods for inactivating a microbe, the methods comprising contacting the microbe with UV light in the presence of riboflavin. In some embodiments, the microbe is a Mycobacterium tuberculosis. Vaccine compositions comprising inactivated microbes, and methods of use thereof, are also provided.

### CROSS-REFERENCE TO RELATED APPLICATIONS

This application claims priority to U.S. Provisional Application Ser. Nos. 63/241,902, filed on Sep. 8, 2021 and 63/086,792, filed on Oct. 2, 2020, each of which is incorporated by reference herein in its entirety for all purposes.

### GOVERNMENT FUNDING

This invention was made with U.S. government support under HHSN272201700018I awarded by the National Institutes of Health and W81XWH-19-1-0223 awarded by the U.S. Army Medical Research and Materiel Command. The government has certain rights in the invention.

### DESCRIPTION OF THE TEXT FILE SUBMITTED ELECTRONICALLY

The contents of the text file submitted electronically herewith are incorporated herein by reference in their entirety: A computer readable format copy of the Sequence Listing, filename: CSUV_013_01WO_SeqList_ST25.txt, date created: Sep. 30, 2021, file size: ≈831 bytes.

### TECHNICAL FIELD

The disclosure relates to inactivated microbial vaccines and methods for preparing the same. More specifically, the disclosure relates to methods for inactivation of microbes using a photosensitizer such as riboflavin in combination with UV light.

### BACKGROUND

Inactivated microbial vaccines are used to protect humans and animals against numerous microbial infections, including typhoid and pertussis. Inactivated microbial vaccines are typically made by exposing microbes to chemical or physical agents, for example, glutaraldehyde, formaldehyde, aldrithiol-2, ethyleneimine derivatives, and irradiation technologies. The use of these inactivating chemistries requires specialized production facilities, specially trained personnel, and cumbersome waste disposal processes. Therefore, these vaccines are difficult to produce in mobile facilities, or under austere environmental conditions. Exposure to harsh chemical and/or physical agents may also destroy the antigenic profile of the microbes, thus reducing or even destroying immunogenicity.

Accordingly, there is a need in the art for compositions and methods for producing effective, inactivated microbial vaccines. In particular, there is a need in the art for compositions and methods for producing an inactivated microbial vaccine against Mycobacterium tuberculosis.

### SUMMARY

The disclosure provides methods for inactivating a microbe, the methods comprising contacting the microbe with a dose of UV light in the presence of riboflavin, wherein the microbe belongs to the genus Mycobacterium. In some embodiments, the microbe is capable of causing disease in a subject. In some embodiments, the subject is a human. In some embodiments, the microbe belongs to the Mycobacterium tuberculosis complex. In some embodiments, the microbe is selected from the group consisting of Mycobacterium tuberculosis, Mycobacterium africanum, Mycobacterium orygis, Mycobacterium bovis, Mycobacterium microti, Mycobacterium canetti, Mycobacterium caprae, Mycobacterium pinnipedii, Mycobacterium suricattae, and Mycobacterium mungi. In some embodiments, the microbe is Mycobacterium tuberculosis. In some embodiments, the microbe is Mycobacterium tuberculosis strain H37Rv. In some embodiments, the microbe is resistant to an antibiotic. In some embodiments, the antibiotic is penicillin, isoniazid, clarithromycin, fluoroquinolone, amikacin, kanamycin, capreomycin, and/or rifamycin.

In some embodiments, the dose of UV light is about 100 Joules to about 25,000 Joules. In some embodiments, the dose of UV light is about 17,000 Joules. In some embodiments, the method comprises altering the genome of the microbe.

In some embodiments, the method comprises selectively oxidizing one or more guanine bases in a nucleic acid of the microbe. In some embodiments, the nucleic acid of the microbe is a DNA or an RNA. In some embodiments, about 1 to about 500 guanine bases in the genome of the microbe are selectively oxidized. In some embodiments, about 1 to about 1000 guanine bases in the genome of the microbe are selectively oxidized. In some embodiments, about 1 to about 2500 guanine bases in the genome of the microbe are selectively oxidized. In some embodiments, about 1 to about 5,000 guanine bases in the genome of the microbe are selectively oxidized. In some embodiments, about 1 to about 10,000 guanine bases in the genome of the microbe are selectively oxidized. In some embodiments, the method does not comprise substantially altering the structure of antigens on the surface of the microbe. In some embodiments, the inactivated microbe is not capable of replicating. In some embodiments, the inactivated microbe is not capable of causing disease in a subject.

The disclosure also provides vaccine compositions comprising a microbe inactivated according to any one of methods disclosed herein. In some embodiments, the composition comprises about 10⁴ microbial cells to about 10⁶ microbial cells. In some embodiments, the composition comprises about 10⁵ microbial cells. In some embodiments, the composition comprises an adjuvant. In some embodiments, the composition comprises a pharmaceutically acceptable carrier or excipient.

The disclosure provides, in some embodiments, vaccine compositions comprising an inactivated Mycobacterium tuberculosis, wherein the Mycobacterium tuberculosis genome comprises one or more oxidized guanine residues. In some embodiments, the Mycobacterium tuberculosis genome comprises about 1-500, about 1-1,000, about 1-5,000, about 1-10,000, or more oxidized guanine bases. In some embodiments, the Mycobacterium tuberculosis is Mycobacterium tuberculosis strain H37Rv. In some embodiments, the antigens present on the surface of the inactivated Mycobacterium tuberculosis microbe are substantially identical to those on the surface of a Mycobacterium tuberculosis microbe that has not been inactivated. In some embodiments, the composition comprises about 10⁴ microbial cells to about 10⁶ microbial cells. In some embodiments, the composition comprises about 10⁵ microbial cells.

In some embodiments, the composition comprises an adjuvant. In some embodiments, the composition comprises a pharmaceutically acceptable carrier or excipient. In some embodiments, the Mycobacterium tuberculosis microbe is inactivated by contacting it with UV light in the presence of riboflavin.

The disclosure further provides methods for treating and/or preventing a mycobacterial infection in a subject in need thereof, the method comprising administering to the subject an effective amount of any one of the vaccine compositions of disclosed herein. In some embodiments, the subject is a mammal. In some embodiments, the subject is a human. In some embodiments, the vaccine is administered intramuscularly. In some embodiments, the vaccine is administered subcutaneously. In some embodiments, the method comprises administering a booster dose of the vaccine composition. In some embodiments, the booster dose is administered about 1 week, about 2 weeks, about 3 weeks, about 4 weeks, about 2 months, about 3 months, about 4 months, about 5 months, about 6 months, or about 1 year after administering the vaccine composition.

The disclosure also provides methods for treating and/or preventing a mycobacterial infection in a subject in need thereof, the method comprising administering to the subject a first vaccine composition, comprising an effective amount of a vaccine composition of disclosed herein (e.g., a vaccine composition comprising microbes inactivated using riboflavin and UV light); and a second vaccine composition, comprising an effective amount of a vaccine composition disclosed herein (e.g., a vaccine composition comprising microbes inactivated using riboflavin and UV light). Additionally, the disclosure provides methods for treating and/or preventing a mycobacterial infection in a subject in need thereof, the method comprising administering to the subject a first vaccine composition comprising an effective amount of the attenuated, live Mycobacterium bovis, and a second vaccine composition, comprising an effective amount of a vaccine composition disclosed herein (e.g., a vaccine composition comprising microbes inactivated using riboflavin and UV light). In some embodiments, the number of microbial cells in the first vaccine composition is greater than the number of microbial cells in the second vaccine composition. In some embodiments, the number of microbial cells in the first vaccine composition is less than the number of microbial cells in the second vaccine composition. In some embodiments, the number of microbial cells in the first vaccine composition is about the same as the number of microbial cells in the second vaccine composition. In some embodiments, the second vaccine composition is administered about 1 week, about 2 weeks, about 3 weeks, about 4 weeks, about 2 months, about 3 months, about 4 months, about 5 months, about 6 months, or about 1 year after the first vaccine composition. In some embodiments, the mycobacterial infection is tuberculosis.

The disclosure additionally provides methods for producing a microbial vaccine, the method comprising (i) providing a plurality of microbes, and (ii) inactivating the microbes by contacting them with a dose of UV light in the presence of riboflavin, wherein the microbes are Mycobacterium tuberculosis microbes. In some embodiments, the dose of UV light is about 100 Joules to about 25,000 Joules. In some embodiments, the dose of UV light is about 17,000 Joules. In some embodiments, the method comprises purifying the inactivated microbes. In some embodiments, the microbes are Mycobacterium tuberculosis strain H37Rv.

These and other embodiments will be described in further detail below.

### BRIEF DESCRIPTION OF THE DRAWINGS

FIG. 1A is a graph showing a reduction in the number of colony forming units (CFUs/mL) after treatment with increasing doses of UV light. FIG. 1B is a graph showing a reduction in the number of colony forming units (CFUs/mL) after treatment with increasing doses of the UV light.

FIG. 2 shows photographs of Mycobacterium smegmatis colonies growing in petri dishes. The left panel shows a control sample (untreated M. smegmatis), and the right panel shows M. smegmatis treated with 17,280 J of UV light in the presence of riboflavin.

FIG. 3 is a graph showing changes in the absorbance ratio (570/600 nm) over time in M. tuberculosis cells treated with 8640 J or 17280 J UV light in the presence of riboflavin, and assayed for growth using alamarBlue® (Thermo Fisher®).

FIG. 4A-4B provides post-infection readouts in lung (FIG. 4A) and spleen (FIG. 4B) from mice in various vaccination groups. Colony forming units (CFUs) are expressed as Log₁₀ CFU of M. Tuberculosis in lung and spleen at day 30 post infection. P value was determined using the one-way analysis of variance (ANOVA), with Tukey post-test; two-tailed unpaired tests were used for statistical comparison between groups using the log 10 CFU-transformed data. **p<0.01, ***p<0.001, ****p<0.0001.

FIG. 5 provides a heatmap showing relative levels of expression of lung cytokines, at day 30 post-infection.

FIG. 6A-6Q show expression levels of individual cytokines in various vaccination groups, including IL-2 (FIG. 6A), IL-5 (FIG. 6B), IL-6 (FIG. 6C), IL-12p70 (FIG. 6D), KC (FIG. 6E), IL-4 (FIG. 6F), IL-17A (FIG. 6G), GM-CSF (FIG. 6H), TNF-alpha (FIG. 6I), IFN-gamma (FIG. 6J), IL-1beta (FIG. 6K), IL-22 (FIG. 6L), IL-113 (FIG. 6M), TSLP9 (FIG. 6N), IL-10 (FIG. 6O), IL-21 (FIG. 6P), IL-alpha (FIG. 6Q).

FIG. 7A-7G show the results from an analysis of post-infection pulmonary pathology in the mouse. H&E stained lung sections were evaluated and assigned a score based on the extent of lung involvement, fibrosis and lesion type. The scoring system used is shown in Table 4.

FIG. 8A-8F show representative H&E stained sections of mouse lungs excised from mice in vaccination groups 1-5.

### DETAILED DESCRIPTION

Tuberculosis (TB) is an infectious disease caused by Mycobacterium tuberculosis (M. Tuberculosis) that is spread through the air from person to person. TB typically affects the lungs, but it can also affect other parts of the body, such as the brain, the kidneys, or the spine. The Bacillus Calmette—Guérin (BCG) vaccine is commonly used to provide protection against TB. The BCG vaccine is prepared using a strain of the attenuated (i.e., virulence-reduced) live bovine tuberculosis bacillus, Mycobacterium bovis, which is not infectious in humans. The vaccine can be administered shortly after birth in infants and produces an immune response that provides partial protection against serious forms of tuberculosis. Because the BCG vaccine is produced using different genetic strains of Mycobacterium bovis, the effectiveness of the vaccine can vary based on geography and the conditions used to grow the various strains. Moreover, because the BCG vaccine comprises live bacteria, its use in some settings is controversial.

The methods described herein can be used to produce improved M. tuberculosis vaccine compositions. The compositions comprise inactivated M. tuberculosis microbes, which retain substantially all of the same antigens as a non-attenuated microbe.

The disclosure provides methods for inactivating M. tuberculosis and other microbes, the methods comprising contacting the microbes with a dose of UV light in the presence of riboflavin. The disclosure also provides inactivated M. tuberculosis and other microbes produced by the methods disclosed herein, compositions comprising these inactivated microbes, and methods of use thereof in treating and/or preventing bacterial infections.

The compositions and methods disclosed herein take advantage of a unique property of the photosensitizer riboflavin and UV light to selectively inactivate M. tuberculosis and other microbes by directed damage to nucleic acids, while preserving the integrity of the proteins and other microbial antigens. The methods and compositions disclosed herein have several advantages compared to compositions and methods currently used in the art. For example, riboflavin is inexpensive, non-toxic and does not pose safety or environmental concerns, allowing for vaccine production in an austere environment with minimal infrastructure and personnel training needs. Additionally, the disclosed inactivation methods are more likely to preserve labile and sensitive antigen profiles that could be destroyed by the methods commonly used in the art. Also, the microbe is inactivated in situ in its native form. The processes described herein inactivate nucleic acid replication without requiring additional processing steps to eliminate the replication potential of the microbe, and do not require extended protein stability throughout processing.

Mycobacterial cells are slow growing and tend to clump together, making their inactivation challenging. In inactivation techniques used in the art, including high energy gamma irradiation, are often used to inactivate bacterial cells. However, such irradiation tends to destroy the antigenic profile of microbes. In contrast, the methods disclosed here, which use UV light having lower energy than gamma irradiation, are surprisingly effective in inactivating microbes in the presence of riboflavin, while also preserving the antigenic profile of the microbes.

### Definitions

The following terms are used in the description herein and the appended claims:

The singular forms “a,” “an” and “the” are intended to include the plural forms as well, unless the context clearly indicates otherwise.

Furthermore, the term “about” as used herein when referring to a measurable value such as an amount, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the terms “reduce,” “reduces,” “reduction” and similar terms mean a decrease of at least about 10%, about 15%, about 20%, about 25%, about 35%, about 50%, about 75%, about 80%, about 85%, about 90%, about 95%, about 97% or more.

As used herein, the terms “enhance,” “enhances,” “enhancement” and similar terms indicate an increase of at least about 10%, about 15%, about 20%, about 25%, about 50%, about 75%, about 100%, about 150%, about 200%, about 300%, about 400%, about 500% or more.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the onset and/or progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the disclosure. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present disclosure.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A human subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant.

“Effective amount” as used herein refers to an amount that, when administered to a subject for treating or preventing a disease (e.g., a microbial infection), or at least one of the clinical symptoms of a disease, is sufficient to affect such treatment or prevention of the disease or symptom thereof. The “effective amount” may vary depending, for example, on the disease and/or symptoms of the disease, severity of the disease and/or symptoms of the disease or disorder, the age, weight, and/or health of the subject to be treated, and the judgment of the prescribing physician. An appropriate amount in any given instance may be ascertained by those skilled in the art or capable of determination by routine experimentation.

Unless otherwise indicated, percent identity is determined herein using the BLAST algorithm available on the World Wide Web at the following address: blast.ncbi.nlm.nih.gov/Blast.cgi.

As used herein, the term “microbe” (interchangeably used with “microbial cell” or “microorganism”) refers to a microscopic organism. In some embodiments, the microbe exists in its single-celled form, while in some embodiments, it exists in a colony of cells. In some embodiments, the microbe is a bacteria, a fungus or a parasite. In some embodiments, the microbe is associated with, or is capable of causing a disease or an infection in a subject.

The term “native” or “wild type” refers to the typical form of a microbe, antigen, protein, or characteristic as found in nature, as distinguished from forms that have been treated, modified and/or processed in any manner. In some embodiments, the native or wild type microbe is capable of replicating, and/or causing disease or an infection in a subject.

As used herein, the term “inactivated” refers to microbes, or a vaccine comprising microbes, that has been treated and/or modified so that the microbes do not have disease-producing capacity. In some embodiments, an inactivated vaccine comprises microbes that have been killed by physical and/or chemical processes. In some embodiments, the inactivated microbes do not replicate in a subject. In some embodiments, the inactivated microbes are alive, while in some embodiments, the inactivated microbes are dead.

As used herein, the terms “immunogen,” “antigen,” and “epitope” refer to substances such as proteins and peptides that are capable of eliciting an immune response.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein, a “vaccine” or “vaccine composition” or an “immunogenic composition” is a composition, which is used to induce an immune response against the microbe that provides protective immunity (e.g., immunity that protects a subject against infection with the pathogen and/or reduces the severity of the disease or condition caused by infection with the pathogen). The protective immune response may include formation of antibodies and/or a cell-mediated response. In some embodiments, the vaccine composition comprises an antigen derived from a microbe.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

### Methods of Producing Inactivated Microbial Vaccines

The disclosure provides methods of inactivating microbes using photochemical technology. This is achieved using photosensitizers that can act as electron transfer agents. The application of photosensitizer agents that can be placed into an excited state in proximity to a guanine base in DNA or RNA constructs allows for selective modification (e.g. oxidation, cross-linking, fragmentation, deamination) of these bases. Because electron chemistry can only occur over short distances, the photosensitizer agent must be bound or associated with (e.g., intercalated with) the nucleic acid in order to carry out the desired chemistry.

In some embodiments, the methods comprise contacting the microbe with a dose of light in the presence of a photosensitizer. In some embodiments, the microbes are added to a solution containing the photosensitizer, or the photosensitizer is added to a solution containing the microbes. In some embodiments, the microbes are cultured, grown, or stored in the presence of the photosensitizer. In some embodiments, the photosensitizer is added to the growth media of the microbes. In some embodiments, the photosensitizer is added to the storage media of the microbes.

In some embodiments, a solution containing the photosensitizer and the microbes (optionally, in media) is subjected to light treatment. In some embodiments, the microbes are pre-incubated for a predetermined period of time in the solution containing the photosensitizer before subjecting the microbes to the light treatment. In some embodiments, the microbes are cultured for a predetermined period of time in media containing the photosensitizer before subjecting the microbes to the light treatment.

In some embodiments, the methods further comprise a step of breaking up clumps or aggregates of microbial cells, and/or of preventing clumping or aggregation of microbial cells. Clumping may be prevented and/or disrupted using any physical technique known in the art, for example, by sonicating, vortexing, or pipetting; using any chemical means known in the art, such as using chelators such as EDTA (ethylenediaminetetraacetic acid) and citrate; and/or using any biological means known in the art, such as using an enzyme (e.g., a peptidase, for example, trypsin). In some embodiments, the step of breaking up clumps of microbial cells, and/or preventing clumping of microbial cells may be performed before and/or after the addition of the photosensitizer. In some embodiments, the step of breaking up clumps of microbial cells, and/or to preventing clumping of microbial cells may be performed in the presence of the photosensitizer.

In some embodiments, the solution comprising microbial cells further comprises an additive that protects cells from hydrodynamic damage. In some embodiments, the additive is a detergent. The type of detergent is not limited, and may be any detergent that is used in the presence of cells. In some embodiments, the detergent is a non-ionic detergent. In some embodiments, the detergent is Pluronic® F-68. In some embodiments, the additive is polyethylene glycol (PEG).

In some embodiments, the photosensitizer is a flavin, for example riboflavin (Vitamin B2), flavin mononucleotide, or flavin adenine dinucleotide. In some embodiments, the photosensitizer is a tertiary aliphatic amine (e.g., 1,4-diazabicyclo(2,2,2)octane), a piperazine, (e.g., N-2-hydroxyethylpiperazine-N′-2-ethanesulfonic acid and 1,4-dimethylpiperazine), an amino acid (e.g., tyrosine, tryptophan, histidine, methionine), an enzyme (e.g., superoxide dismutase) or EDTA. In some embodiments, the photosensitizer is riboflavin.

In some embodiments, the concentration of photosensitizer used during inactivation is about 10 μM to about 1 mM, for example, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 200 μM, about 300 μM, about 400 μM, about 500 μM, about 600 μM, about 700 μM, about 800 μM, about 900 μM, or about 1 mM, including all values and subranges therebetween. In some embodiments, the solution or media containing the photosensitizer contains the photosensitizer at a concentration of about 1 μM to about 50 μM, for example, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, or about 50 μM, including all values and subranges therebetween. In some embodiments, the photosensitizer concentration in the solution or media is less than about 10 μM, for example, less than about 9 μM, less than about 8 μM, less than about 7 μM, less than about 6 μM, less than about 5 μM, less than about 4 μM, less than about 3 μM, less than about 2 μM, or less than about 1 μM, including all values and subranges therebetween.

In some embodiments, the light treatment comprises treatment with visible light, ultraviolet light, and/or infrared light. The light treatment inactivates a nucleic acid (e.g., DNA and/or RNA) in the microbes by modifying bases of these nucleic acids. In some embodiments, guanine bases are selectively modified. In some embodiments, guanine bases are selectively oxidized. Oxidized guanine bases cannot be repaired by natural enzymatic and cell repair mechanisms. As such, there is no possibility for reversion of the induced change to a form that would restore the ability of the microbes to cause disease.

In some embodiments, the light treatment comprises treatment with ultraviolet (UV) light. The UV light may be UV-A, UV-B, or UV-C light. The UV light may have a wavelength of 170 to 400 nm, including all values and subranges therebetween. For example, in some embodiments, the UV light has a wavelength of 315 to 400 nm, 310 to 320 nm, 280 to 360 nm, 280 to 315 nm, or 180 to 280 nm. The UV light may be provided by UV light sources known in the art, such as the Mirasol® PRT Illumination device (TerumoBCT, Lakewood, Colorado). In some embodiments, the microbes may be treated with multiple wavelengths of light simultaneously.

In some embodiments wherein riboflavin is used as a photosensitizer, UV light having a wavelength of 310 to 320 nm is used. Without being bound by any theory, it is believed that this wavelength prevents riboflavin from reacting in free solution, which would result in production of undesirable oxygen free radicals. At these wavelengths, riboflavin will react only when intercalated with nucleic acid.

The dose of the UV light may vary depending on the volume of solution being treated. In some embodiments, the dose of the UV light is in the range of about 100 Joules to about 50,000 Joules, for example, about 200 Joules, about 300 Joules, about 400, about 500, about 600 Joules, about 700 Joules, about 800 Joules, about 900 Joules, about 1000 Joules, about 2000 Joules, about 3000 Joules, about 4000 Joules, about 5000 Joules, about 6000 Joules, about 7000 Joules, about 8000 Joules, about 9000 Joules, about 10,000 Joules, about 11,000 Joules, about 12,000 Joules, about 13,000 Joules, about 14,000 Joules, about 15,000 Joules, about 16,000 Joules, about 17,000 Joules, about 18,000 Joules, about 19,000 Joules, about 20,000 Joules, about 25,000 Joules, about 30,000 Joules, about 35,000 Joules, about 40,000 Joules, about 45,000 Joules, or about 50,000 Joules, including all values and subranges that lie therebetween. In some embodiments, the dose of the UV light is about 17,000 Joules. In some embodiments, the dose of the UV light is about 8,640 Joules. In some embodiments, the dose of the UV light is about 17,280 Joules.

In some embodiments, the volume of microbial preparations for illumination is in the range of about 200 ml to about 600 ml, for example about 200, about 225, about 250, about 275, about 300, about 325, about 350, about 375, about 400, about 425, about 450, about 475, about 500, about 525, about 550, about 575, or about 600 ml, including all values and subranges that lie therebetween. In some embodiments, the volume of microbial preparations for illumination is in the range of about 170 mL to about 370 mL of solution. In some embodiments, the volume of microbial preparations for illumination is about 300 mL.

In some embodiments, the dose of UV light is in the range of about 0.3 Joules/ml to about 170 Joules/ml, for example, about 0.4, about 1.0, about 2.0, about 5, about 10, about 20, about 30, about 40, about 50, about 60, about 70, about 80, about 90, about 100, about 110, about 120, about 130, about 140, or about 150 Joules/ml, including all values and subranges that lie therebetween. In some embodiments, the dose of UV light may be about 30 Joules/ml. In some embodiments, the dose of UV light may be about 60 Joules/ml.

The microbes may be treated with UV light for about 1 minute to about 60 minutes, for example, about 1, about 2, about 3, about 4, about 5, about 6, about 7, about 8, about 9, about 10, about 15, about 20, about 25, about 30, about 35, about 40, about 45, about 50, about 55, or about 60 minutes, including all values and subranges that lie therebetween. In some embodiments, the microbes are treated with UV light for about 1 minute to about 10 minutes, about 1 minute to about 5 minutes, or about 1 minute to about 3 minutes.

In some embodiments, the solution being treated comprises about 10 microbial cells to about 10¹² microbial cells, for example, about 10², about 10³, about 10⁴, about 10⁵, about 10⁶, about 10⁷, about 10⁸, about 10⁹, about 10¹⁰, about 10¹¹, or about 10¹² microbial cells, including all values and subranges that lie therebetween. In some embodiments, the solution being treated comprises about 10⁴ microbial cells to about 10⁶ microbial cells. In some embodiments, the solution being treated comprises about 10⁵ microbial cells. In some embodiments, the microbe is a bacterium, a fungus, or a parasite. In some embodiments, the microbe is capable of infecting a subject, for example, a human subject.

In some embodiments, the microbe is a bacterium. In some embodiments, the bacterium is selected from the group consisting of Actinomyces israelii, Acinetobacter baumanii, Bacillus anthracis, Burkholderia cepacia, Bacterioides fragilis, Borrelia burgdorferi, Borrelia garinii, Borrelia afzelii, Borrelia recurrentis, Brucella abortus, Brucella canis, Brucella melitensis, Brucella suis, Bordetella pertussis, Bacillus subtilis, Chlamydia pneumoniae, Chlamydia trachomatis, Chlamydophila psittaci, Citrobacter freundii, Campylobacter jejuni, Clostridium botulinum, Clostridium difficule, Clostridium neoformans, Clostridium perfringens, Clostridium tetani, Corynebacterium diphtheria, Ehrlichia canis, Ehrlichia chaffeensis, Enterococcus faecalis, Enterococcus faecium, Escherichia coli, Enterobacter aerogenes, Enterobacter cloacae, Francisella tularensis, vancomycin-resistant Enterococci (VRE), Haemophilus influenzae, Helicobacter pylori, Klebsiella oxytoca, Klebsiella pneumonia, Legionella pneumophila, Leptospira alexanderi, Leptospira borgpetersenii, Leptospira interrogans, Leptospira kirschneri, Leptospira mayottensis, Leptospira noguchii, Leptospira santarosai, Leptospira weilii, Listeria monocytogenes, Mycobacterium avium, Mycobacterium tuberculosis, Mycobacterium leprae, Mycoplasma pneumonia, Nocardia farcinica, Neisseria meningitides, Neisseria gonorrhoeae, Nocardia asteroids, Pseudomonas aeruginosa, Propionibacterium acnes, Rickettsia rickettsia, Salmonella typhi, Salmonella paratyphi, Salmonella typhimurium, Serratia marcescens, Shigella flexneri, Shigella sonnei, Shigella dysenteriae, Stenotrophomonas maltophilia, Staphylococcus aureus, methicillin-susceptible Staphylococcus aureus (MSSA), methicillin-resistant Staphylococcus aureus (MRSA), Staphylococcus epidermidis, Staphylococcus saprophyticus, Streptococcus agalactiae, Streptococcus pneumoniae, Streptococcus viridans, Streptococcus pyogenes, Streptococcus Group A, Streptococcus Group B, Streptococcus Group C, Treponema pallidum, Vibrio cholera, Yersinia pestis and Yersinia pseudotuberculosis.

In some embodiments, the microbe is a fungus. In some embodiments, the fungus is selected from the group consisting of Aspergillus fumigatus, Aspergillus flavus, Aspergillus clavatus, Blastomyces dermatitidis, Basidiobolus ranarum, Candida albicans, Candida glabrata, Candida guilliermondii, Candida krusei, Candida lusitaniae, Candida tripicalis, Candida parpasilosis, Cryptococcus neoformans, Cryptococcus gattii, Coccidioides immitis, Coccidioides posadasii, Conidiobolus coronatus, Conidiobolus incongruous, Cladophialphora bantianum, Rhinocladiella mackenziei, Dactylaria gallopava, Fusarium solani, Fusarium oxysporum, Fusarium verticillioides, Fusarium proliferatum, Exophiala jeanselmei, Exophiala dermatitidis, Curvularia lunata, Bipolaris spicifera, Bipolaris hawaiiensis, Bipolaris cynodontis, Alternaria alternata, Histoplasma capsulatum, Histoplasma duboisii, Histoplasma farciminosum, Lacazia loboi, Mucor indicus, Malassezia furfur, Trichosporon asahii, Pneumocystis jirovecii, Pneumocystis carinii, Paracoccidioides brasiliensis, Pseudoallescheria boydii, Paracoccidioides lutzii, Penicillium marneffei, Penicillium aurantiogriseum, Sporothrix schenkii, Stachybotrys chartarum, Saccharomyces cerevisiae, Sporothrix brasiliensis, Sporothrix schenckii, and Sporothrix globose.

In some embodiments, the microbe is a parasite. In some embodiments, the parasite is selected from the group consisting of Ancylostoma ceylanicum, Ancylostoma duodenale, Acanthamoeba culbertsoni, Acanthamoeba polyphaga, Acanthamoeba castellanii, Acanthamoeba astronyxis, Acanthamoeba hatchetti, Acanthamoeba rhysodes, Acanthamoeba divionensis, Acanthamoeba lugdunensis, Acanthamoeba lenticulata, Ancylostoma duodenale, Angiostrongylus cantonensis, Brugia malayi, Balamuthia mandrillaris, Babesia microti, Balantidium coli, Blastocystis hominis, Cyclospora cayetanensis, Cryptosporidium, Dientamoeba fragilis, Entamoeba histolytica, Entamoeba coli, Entamoeba dispar, Entamoeba hartmanni, Entamoeba polecki, Fasciola hepatica, Fasciola magna, Fasciola gigantic, Giardia lamblia, Isospora belli, Leishmania donovani, Leishmania infantum, Leishmania mexicana, Enterobius vermicularis, Ascaris lumbricoides, Trichuris trichuria, Necator americanus, Naegleria fowleri, Onchocerca volvulus, Dracanculus medinensis, Trichinella spiralis, Strongyloides steroralis, Clonorchis sinensis, Paragonimus westermani, Plasmodium falciparum, Plasmodium vivax, Plasmodium malariae, Plasmodium ovale curtisi, Plasmodium ovale wallikeri, Plasmodium malariae, Plasmodium knowlesi, Schistosoma mansonii, Schistosoma haematobium, Schistosoma japonicum, Schistosoma mekongy, Taenia saginata, Taenia solium, Trichomonas vaginalis, Trypanosoma brucei, Trypanosoma cruzi, Toxoplasma gondii and Wuchereria bancrofti.

In some embodiments, the microbe belongs to the genus Mycobacterium. In some embodiments, the microbe belongs to the Mycobacterium tuberculosis complex. In some embodiments, the microbe is selected from the group consisting of Mycobacterium tuberculosis, Mycobacterium africanum, Mycobacterium orygis, Mycobacterium bovis, Mycobacterium microti, Mycobacterium canetti, Mycobacterium caprae, Mycobacterium pinnipedii, Mycobacterium suricattae, and Mycobacterium mungi. In some embodiments, the microbe is Mycobacterium tuberculosis. In some embodiments, the microbe is the Erdman strain, the H37Rv strain, or the CDC1551 strain of Mycobacterium tuberculosis. In some embodiments, the microbe is the H37Rv strain of Mycobacterium tuberculosis.

In some embodiments, the microbe is Mycobacterium leprae, Mycobacterium lepromatosis, or nontuberculous Mycobacteria (NTM). In some embodiments, the microbe belongs to the Mycobacterium avium complex. In some embodiments, the microbe is selected from the group consisting of Mycobacterium avium, Mycobacterium avium paratuberculosis, Mycobacterium avium silvaticum, Mycobacterium avium hominissuis, Mycobacterium colombiense, Mycobacterium indicus pranii, and Mycobacterium intracellulare.

In some embodiments, the microbe is resistant to an antibiotic. In some embodiments, the antibiotic is penicillin, isoniazid, clarithromycin, fluoroquinolone, amikacin, kanamycin, capreomycin, and/or rifamycin.

In some embodiments, the photosensitizer enters the microbial cell in a passive manner. In some embodiments, the photosensitizer is imported into the microbes by pinocytosis, or clathrin-mediated endocytosis. In some embodiments, the microbe actively imports the photosensitizer. In some embodiments, the microbe is genetically engineered to import an amount of photosensitizer that is higher than the amount of photosensitizer imported into a wild type microbial cell under the same conditions. In some embodiments, the microbe is genetically engineered to overexpress one or more receptors of the photosensitizer. In some embodiments, the microbe comprises a polynucleotide encoding one or more receptors of the photosensitizer.

In some embodiments, the microbe is genetically engineered to overexpress one or more receptors of riboflavin. In some embodiments, the microbe comprises a polynucleotide encoding one or more receptors of riboflavin. The receptor of riboflavin is not limited, and may be any receptor known in the art, or discovered in the future, to import riboflavin into a microbial cell. In some embodiments, the receptor is RibM (GenBank Accession No: K4REQ6), RibN (GenBank Accession No: Q1MIM3), RfuABCD (GenBank Accession No: Q56328), RibU (GenBank Accession No: Q5M614), ImpX (GenBank Accession No: D5RAW5), RfnT (GenBank Accession No: A6X7E7), or RibV (GenBank Accession No: Q6F0N9), or a functional ortholog or variant thereof. In some embodiments, the receptor is any one of the bacterial riboflavin receptors described in Gutiérrez-Preciado A, et al., PLoS One. 2015; 10(5), which is incorporated herein by reference in its entirety.

In some embodiments, the microbes are not subjected to any additional purification or modification steps after light treatment. In other embodiments, the microbes are purified after the light treatment. In some embodiments, the microbes are concentrated after the light treatment. In some embodiments, the photosensitizer is removed from the solution containing the microbes after light treatment.

In some embodiments, the microbes are combined with one or more additional pharmaceutically acceptable carriers after light treatment. In some embodiments, the microbes are combined with a solution comprising an adjuvant after light treatment.

In some embodiments, the method comprises selectively oxidizing guanine bases in a nucleic acid of the microbe. Without being bound by any theory, it is believed that the inactivation of the microbes is, at least in part, associated with, caused by, or results from the selective oxidation of guanine bases in a nucleic acid of the microbe. In some embodiments, the method comprises selectively oxidizing at least about 1 to at least about 100,000 guanine bases in a nucleic acid (e.g., a DNA or an RNA) of the microbe, for example at least 10, at least 100, at least 1000, at least 5000, at least 10,000, at least 15,000, at least 20,000, at least 50,000, at least 75,000, at least 100,000 or more guanine bases, including all subranges and values that lie therebetween. In some embodiments, about 1%, about 2%, about 3%, about 4%, about 5%, about 6%, about 7%, about 8%, about 9%, about 10%, about 20%, about 40%, about 60%, about 80%, or about 100% of the guanine bases in the genome of the microbe are oxidized. In some embodiments, the inactivation process does not substantially change the phenotype or structure of the microbes, or the phenotype or structure of the antigens on the surface of the microbes.

In some embodiments, the method comprises contacting Mycobacterium tuberculosis H37Rv cells with about 8,640 J of UV light in the presence of about 50 μM of riboflavin in a volume of about 300 mL. In some embodiments, the method comprises contacting Mycobacterium tuberculosis H37Rv cells with about 17,280 J of UV light in the presence of about 50 μM of riboflavin in a volume of about 300 mL. In some embodiments, the method further comprises sonication of the Mycobacterium tuberculosis cells.

### Inactivated Microbial Vaccine Compositions

The disclosure provides the inactivated microbes produced by any one of the methods disclosed herein, and compositions comprising the inactivated microbes disclosed herein. In some embodiments, the compositions comprise inactivated microbes, or fragments or derivatives thereof. In some embodiments, the compositions comprise inactivated microbes of more than one species, for example, the composition may comprise inactivated M. tuberculosis and inactivated M. leprae.

In some embodiments, the inactivated microbes substantially maintain and preserve the antigen and epitope profile, microbe integrity, and protein and/or lipid structure of the original, native microbe or antigen. In some embodiments, the inactivated microbes are not capable of replicating. In some embodiments, the inactivated microbes have modified genome (that is, modified DNA and/or RNA). In some embodiments, the DNA and/or RNA of the inactivated microbes comprises modified bases. In some embodiments, the modification of the microbial DNA and/or RNA renders the microbe incapable of replicating. In some embodiments, the modification of the microbial DNA and/or RNA does not kill the microbe. For example, in some embodiments, the DNA or RNA of the inactivated microbes may comprise modified guanine bases, such as oxidized guanine bases. In some embodiments, an inactivated microbe comprises about 1 to about 100,000 modified guanine bases, for example about 10, about 100, about 1000, about 5000, about 10,000, about 15,000, about 20,000, about 50,000, about 75,000, about 100,000 or more modified guanine bases, including all subranges and values that lie therebetween, in its genome.

In some embodiments, the compositions disclosed herein do not comprise any adjuvant. In some embodiments, the compositions further comprise an adjuvant. In some embodiments, the adjuvant boosts the immunological response. For example, in some embodiments, the adjuvant modifies monocyte function.

A non-limiting list of adjuvants includes saponin formulations, virosomes, microbe-like particles, non-toxic derivatives of enterobacterial lipopolysaccharide (LPS), immunostimulatory oligonucleotides (e.g., an immunostimulatory oligonucleotide containing a CpG motif), mineral containing compositions, oil-emulsions, polymers, micelle-forming adjuvants (e.g., a liposome), immunostimulating complex matrices (e.g., ISCOMATRIX), particles, squalene, phosphate, cationic liposome-DNA complexes (CLDC), DDA, DNA adjuvants, gamma-insulin, ADP-ribosylating toxins, detoxified derivatives of ADP-ribosylating toxins, Freund's complete adjuvant, Freund's incomplete adjuvant, muramyl dipeptides, monophosphoryl Lipid A (MPL), poly IC, CpG oligodeoxynucleotides (ODNs), imiquimod, adjuvant system AS01, adjuvant system AS02, adjuvant system AS03, MF59® (i.e., an oil-in-water emulsion containing metabolizable oil squalene and the surfactants Tween 80 and sorbitan trioleate Span 85) and aluminum or aluminum salts (e.g., alum, aluminum phosphate, aluminum hydroxide). In some embodiments, a CpG ODN is a class A, class B or a class C CpG ODN. In some embodiments, a CpG ODN is CpG 7909 (InvivoGen, San Diego, CA). Other suitable adjuvants include TLR agonists, NOD agonists, and lipid-DNA agonist complexes. In some embodiments, the adjuvant is GLA-SE, which is a synthetic toll-like receptor 4 agonist (see, e.g., Behzad, H. et al, J Infect Dis., 2021, 205(3): 466-473). In some embodiments, the adjuvant comprises GLA-SE in a squalene emulsion.

In some embodiments, the adjuvant is capable of promoting a Th1-type immune response. In some embodiments, the adjuvant is capable of limiting a Th2-type response. In some embodiments, the adjuvant is CpG and/or AS01. In some embodiments, the adjuvant is a phosphorothioate oligonucleotide comprising about 15 to about 30 nucleotides. In some embodiments, the adjuvant comprises a nucleic acid that comprises the sequence 5′-TGACTGTGAACGTTCGAGATGA-3′ (SEQ ID NO: 1). In some embodiments, the adjuvant comprises a nucleic acid that comprises the sequence 5′-TCCATGACGTTCCTGATGCT-3′ (SEQ ID NO: 2). In some embodiments, the adjuvant is ODN 1668. In some embodiments, the adjuvant is CpG 1018. In some embodiments, the composition comprises a pharmaceutically acceptable carrier or excipient.

When used herein to refer to an adjuvant, the term “capable of promoting a Th1-type immune response” refers to those adjuvants that promote a Th1-type response over a Th2-type response. Th1-type and Th2-type immune responses are distinguished by the types of immune cells involved and the cytokines produced thereby. T helper type 1 (Th1) lymphocytes secrete interleukin (IL)-2, interferon-γ, and lymphotoxin-α and stimulate type 1 immunity, which is often characterized by intense phagocytic activity. Conversely, Th2 cells secrete IL-4, IL-5, IL-9, IL-10, and IL-13 and stimulate type 2 immunity, which is often characterized by high antibody titers. Non-limiting examples of adjuvants capable of promoting a Th1-type response over a Th2-type response include CpG and/or AS01, CpG 1018, ODN 1688, or AdVax™. AdVax™ comprises delta inulin, specifically delta inulin of highly specific particle size and morphology (See, e.g., Petrovsky, N., et al., Vaccine; 33(44): 5920-5926 (2015)). Not all adjuvants can promote a Th1-type immune response. For example, Montanide™ is one type of adjuvant that does not promote a Th1-type immune response (See, e.g., van Doorn, E., et al., Hum Vaccin Immunother; 12(1): 159-169 (2016)).

In some embodiments, the inactivated microbial vaccine composition further comprises one or more agonists or antagonists. In some embodiments, the agonist is a Toll-Like Receptor (TLR) agonist. In some embodiments, the TLR agonist is an agonist of one or more of TLR1, TLR2, TLR3, TLR4, TLR5, TLR6, TLR7, TLR8, TLR9, TLR10, TLR11, or TLR12. In some embodiments, the agonist is a TLR3 and/or a TLR9 agonist. In some embodiments, the antagonist is a C-C chemokine receptor type 2 (CCR2) antagonist. In some embodiments, the antagonist is an angiotensin receptor blocker (ARB), such as losartan, telmisartan, irbesartan, azilsartan, candesartan, eprosartan, olmesartan, or valsartan. In some embodiments, the ARB is administered at a dose of between about 5 and about 100 mg/kg, for example about 5, about 10, about 15, about 20, about 25, about 30, about 35, about 40, about 45, about 50, about 55, about 60, about 65, about 70, about 75, about 80, about 85, about 90, about 95, or about 100 mg/kg, including all values and subranges that lie therebetween.

In some embodiments, the inactivated microbe vaccine comprises at least one of (i.e., one of, two of, or all three of) a TLR agonist, a CCR2 antagonist and an ARB.

In some embodiments, the agonist or antagonist (e.g., a TLR3 and/or a TLR9 agonist) is contained within or coupled to a liposome. Liposomes are spherical, self-enclosed vesicles composed of amphipathic lipids. Liposomes may be unilamellar, having one lipid bilayer membrane, or multilamellar, having two or more concentrically arranged bilayers. Suitable liposomes may have a selected mean particle size diameter of about 200-500 nm. Various methods of preparing liposomes and encapsulation of therapeutic agents therein are well documented (see, for example, U.S. Pat. Nos. 3,932,657, 4,311,712, and 5,013,556, all of which are incorporated herein by reference). Known methods include the reverse phase evaporation method as described in U.S. Pat. No. 4,235,871, which is incorporated herein by reference.

Lipids for use in forming the liposomes described herein include vesicle-forming lipids having two hydrocarbon chains, typically acyl chains, and a polar head group. Included in this class are the phospholipids, such as phosphatidylcholine (PC), phosphatidylethanolamine (PE), phosphatidic acid (PA), phosphatidylinositol (PI), and sphingomyelin (SM), where the two hydrocarbon chains are typically between about 14-22 carbon atoms in length, and have varying degrees of unsaturation. The selection of lipids and proportions can be varied to achieve a desired degree of fluidity or rigidity, to control stability, and/or to control the rate of release of an entrapped agent. Where more than one type of lipid is used, a suitable amount of a relatively unsaturated lipid (such as PC), may be used in order to form stable liposomes. In one embodiment, at least 45-50 mol % of the lipids used to form the liposome are PC.

The liposomes may also include lipids derivatized with a hydrophilic polymer such as polyethylene glycol (PEG). Suitable hydrophilic polymers include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide, and hydrophilic peptide sequences. Methods of preparing lipids derivatized with hydrophilic polymers are known (see e.g., U.S. Pat. No. 5,395,619, which is incorporated herein by reference).

In some embodiments, the inactivated microbial vaccine comprises cationic liposome-DNA complexes (CLDC).

In some embodiments, the inactivated microbial vaccine further comprises a photosensitizer such as riboflavin (vitamin B2). In some embodiments, the inactivated microbial vaccine is substantially free of photosensitizer.

In some embodiments, the inactivated microbial vaccine composition further comprises a carrier. In some embodiments, the cells and/or the photosensitizer are suspended in the carrier. In some embodiments, the carrier comprises normal saline (e.g., 0.9% sodium chloride), dextrose saline (e.g., dextrose 5% in 0.9% sodium chloride), phosphate buffered saline (e.g., 137 mmol/L NaCl, 2.7 mmol/L KCl, 10 mmol/L Na₂HPO₄, 2 mmol/L KH₂PO₄).

In some embodiments, the inactivated microbial vaccine composition further comprises one or more additional pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilizers, solubilizers, surfactants (e.g., wetting agents), masking agents, coloring agents, flavoring agents, and sweetening agents. Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994, each of which is incorporated herein by reference in its entirety.

In some embodiments, an inactivated microbe is associated with and/or contained within a liposome, virosome, ISCOM (immunostimulatory complex) or virus-like particle. These types of delivery systems offer numerous advantages, including one or more of the following: i) protection of the inactivated microbe against enzymes, ii) increased stability of the inactivated microbe during vaccine manufacturing, transport, and storage, iii) increased efficacy of presentation of antigens on the inactivated microbe to antigen presenting cells (APCs), and iv) increased half-life of the inactivated microbe after administration to a subject.

In some embodiments, an inactivated microbe is associated with and/or contained within a nanoparticle. In some embodiments, an inactivated microbe is associated with and/or contained within a Gantrez Nanoparticle (GNP). See, e.g., Gomez et al, Journal of Immunological Methods 348 (2009) 1-8; Gomez et al, Vaccine 25 (2007) 5263-5271. GNPs comprise a copolymer of methyl vinyl ether and maleic anhydride, which readily reacts with amino groups, making it easy to load or link different proteins thereto. Methods for production of GNPs are known, such as a solvent displacement method. The inactivated microbe may be incorporated into the GNP during manufacture (i.e., embedded in the nanoparticle), or after the preparation of the GNP (i.e., coating the nanoparticle). In some embodiments, the nanoparticle is a poly(anhydride) nanoparticle. See, e.g., Irache et al, Frontiers in Bioscience S2, 876-890, 2010. In some embodiments, the nanoparticle has a diameter in the range of about 50 to about 500 nm, such as about 50, about 75, about 100, about 125, about 150, about 175, about 200, about 225, about 250, about 275, about 300, about 325, about 350, about 375, about 400, about 425, about 450, or about 500 nm. In some embodiments, the nanoparticle further comprises an adjuvant.

In some embodiments, an inactivated microbe is associated with and/or contained within a microparticle. In some embodiments, an inactivated microbe is associated with and/or contained within a poly-ε-caprolactone (PCL) microparticle. See, e.g., Roban, B.S. et al, Clin Exp Allergy. 2007 February; 37(2):287-95. In some embodiments, the microparticle has a diameter of about 1 to about 3 μm, such as about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9 or about 3.0 μm. In some embodiments, the microparticle further comprises an adjuvant.

In some embodiments, an inactivated microbe is associated with a polymer. In some embodiments, an inactivated microbe is encapsulated by a polymer. The polymer may be, for example, a polymer that enhances the stimulation of mucosal immunity. In some embodiments, the polymer is a biodegradable polymer. For example, the biodegradable polymer may be a copolymer of methyl vinyl ether and maleic anhydride (PVM/MA). In some embodiments, the copolymer has a molecular weight between 100 and 2400 kDa, such as between 200 and 2000 kDa or between 1880 and 250 kDa. Illustrative polymers are described in U.S. Pat. Nos. 8,628,801, 9,522,197 and 10,933,025, which are incorporated by reference in their entireties.

In some embodiments, the polymer is a mucoadhesive polymer. In some embodiments, the polymer is a chitosan-based mucoadhesive polymer such as chitosan-cysteine, chitosan-4-thio-butylamidine, chitosan-thioglycolic acid, chitosan-glutathione, chitosan-6-mercaptonicotinic acid, chistoan-N-acetylcysteine, chitosan-4-mercaptobenzoic acid, or chitosan-N-acetylpenicillamine. In some embodiments, the polymer is a eudragit-based mucoadhesive polymer. In some embodiments, the polymer is a hybrid polymer, such as poly (lactic-co-glycolic acid) (PLGA). In some embodiments, the polymer may allow for more efficient delivery of the inactivated microbe using an oral or nasal route, as compared to a microbe that is not encapsulated by a polymer.

In some embodiments, a vaccine composition may comprise an inactivated microbe and a PLGA copolymer, such as poly(lactide) homopolymer (PLA) or poly(lactic-co-glycolic acid) copolymer (PLGA). In some embodiments, the inactivated microbe is comprised in a PLGA nanoparticle.

### Methods of Treatment

In some embodiments, the compositions described herein can be used to elicit an immune response against the inactivated microbe in subjects. In some embodiments, the immune response is a Th2 immune response. In some embodiments, the immune response is a Th1 immune response. In some embodiments, the compositions described herein are vaccine compositions.

The disclosure provides methods of eliciting an immune response in a subject in need thereof, the method comprising administering to the subject an effective amount of any one of the inactivated microbes, or any one of the compositions disclosed herein. The induction of the immune response has the ability to prevent and/or treat a disease associated with or caused by the native microbe. Therefore, the disclosure also provides methods for treating and/or preventing a microbial infection in a subject in need thereof, the method comprising administering to the subject an effective amount of any one of the inactivated microbes, or any one of the compositions disclosed herein. As used herein, a “microbial infection” is a disease or infection associated with, resulting from, or caused by a microbe, such as any one or more microbes described herein.

The disease may be any disease or infection caused by any one of the microbes described herein. In some embodiments, the disease is a mycobacterial infection. As used herein, a “mycobacterial infection” is a disease or infection associated with, resulting from, or caused by a bacteria belonging to the genus Mycobacteria. In some embodiments, the mycobacterial infection is a pulmonary infection. In some embodiments, the mycobacterial infection is selected from the group consisting of tuberculosis, leprosy, and nontuberculous pulmonary disease. In some embodiments, the tuberculosis is drug resistant tuberculosis. In some embodiments, the drug resistant tuberculosis is multidrug-resistant tuberculosis (MDR TB), or extensively drug resistant tuberculosis (XDR TB).

In some embodiments, the subject is assessed for immune function and immune status prior to administration of the vaccine. Such assessments may include, but are not limited to, DTH skin testing, blood tests, lymph node aspirate tests, tumor tissue tests, and/or determination of whether the subject is anergic, B cell responsive, etc. In some embodiments, the subject is not assessed for immune function and immune status prior to administration of the vaccine. In some embodiments, the subject is immunocompetent. In some embodiments, the subject is immunocompromised. Optionally, the vaccine may be used in combination with genetic testing to quantify the degree of immune-responders, or immune non-responders.

In some embodiments, the vaccine composition does not comprise any adjuvant. In some embodiments, a vaccine composition further comprises an adjuvant. In some embodiments the adjuvant is CpG. In some embodiments, the CpG is CpG 7909. In some embodiments, a vaccine dose comprises from about 25 μg to about 750 μg CpG, or from about 50 μg to about 500 μg CpG.

In some embodiments, the inactivated microbial vaccine is administered once, or more than once to the subject. In some embodiments, the vaccine is administered once, twice, three times, four times, five times, six times, seven times, eight times, nine times, or ten times to the subject.

The inactivated microbial vaccine may be administered to the subject every day, about every 3 days, about every 7 days, about every fourteen days, about once per month, or about once per year. In some embodiments, the vaccine is administered at least once per week, at least every two weeks, or at least once every six months. In some embodiments, the vaccine is administered once, twice, three times, four times, five times, six times, seven times, eight times, nine times, ten times, twelve times, fifteen times, twenty times, or twenty-five times in a year.

In some embodiments, a first dose (e.g., a prime dose) of inactivated microbial vaccine is administered, followed by a second dose (e.g., a booster dose) to the subject. In some embodiments, the second dose is administered about 1 week, about 2 weeks, about 3 weeks, about 4 weeks, about 2 months, about 3 months, about 4 months, about 5 months, about 6 months, or about 1 year after the first dose.

In some embodiments, a first inactivated microbial vaccine and a second inactivated microbial vaccine are administered to the subject. In some embodiments, the first inactivated microbial vaccine and the second inactivated microbial vaccine are different. In some embodiments, the second inactivated microbial vaccine is administered after the first vaccine to boost the immune response. In some embodiments, immune response in the subject is monitored between administration of the first vaccine and the second vaccine. In some embodiments, the second vaccine is administered when it is determined that the subject has not exhibited a satisfactory immune response following administration of the first vaccine.

It will be appreciated by one of skill in the art that appropriate number of microbes in the vaccine composition can vary. For example, in some embodiments, the vaccine composition comprises about 1×10³, about 1×10⁴, about 1×10⁵, about 1×10⁶, about 1×10⁷, about 1×10⁸, about 1×10⁹, or about 1×10¹⁰ microbes. In some embodiments, a vaccine composition comprises about 1×10⁵ to about 1×10⁸ microbes. In some embodiments, a vaccine composition comprises about 1×10⁴ to about 1×10⁶ microbes. In some embodiments, a vaccine composition comprises about 1×10⁵ microbes. In some embodiments, about 1×10⁴ to about 1×10⁸ microbes, for example, about 1×10⁵ microbes are administered to a subject in each administration. For example, about 1×10⁵, about 5×10⁵, about 1×10⁶, about 5×10⁶, about 1×10⁷, about 5×10⁷, or about 1×10⁸ microbes may be administered to a subject per administration. In some embodiments, the administered dose is a split dose, wherein the total number of microbes for administration is divided into 2, 3, 4, 5, 6, 7, 8, 9, or 10 sub-doses.

The inactivated microbial vaccine may be delivered to the subject intramuscularly, intramucosally, intranasally, subcutaneously, intratumorally, intradermally, transdermally, intravaginally, intraperitoneally, intrarectally, intra-articularly, intra-lymphatically, orally or intravenously. In some embodiments, administration may be by sublingual, buccal, intra-organ (e.g., intrasplenic), or inhaled routes. For intravenous, cutaneous or subcutaneous injection, or injection at the site of an infection, the vaccine composition may be in the form of a parenterally acceptable aqueous solution which has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilizers, buffers, antioxidants and/or other additives may be included, as required.

In some embodiments, the vaccine is administered peripherally to the subject. In some embodiments, multiple aliquots of the vaccine are administered peripherally to the subject, in different locations.

One or more sub-dose may be administered to the subject peripherally, at different locations on the subject's body. Each sub-dose may be administered at approximately the same time, or administration of the sub-doses may be staggered. For example, sub-doses may be administered at intervals of 15 minutes, 20 minutes, 30 minutes, 45 minutes, 1 hour, or 3 hours.

In some embodiments, the vaccine is administered simultaneously or sequentially (either before or after) with a vaccine-enhancing agent. In some embodiments, the vaccine-enhancing agent is an angiotensin receptor blocker (ARB) or a beta blocker (BB). Exemplary vaccine-enhancing agents include losartan, telmisartan, irbesartan, azilsartan, candesartan, eprosartan, olmesartan, valsartan, propranolol, acebutolol, atenolol, betaxolol, bisoprolol, carteolol, carvedilol, esmolol, labetalol, metoprolol, nadolol, nebivolol, penbutolol, pindolol, propranolol, sotalol, timolol. In some embodiments, the vaccine-enhancing agent is selected from the group consisting of losartan and propranolol. In some embodiments, the vaccine-enhancing agent is losartan. In some embodiments, the vaccine-enhancing agent is propranolol.

In some embodiments, a method for vaccinating a subject comprises administering an inactivated microbial vaccine composition comprising inactivated microbes, and a potent adjuvant comprising TLR3 and/or TLR9 agonists attached to liposomes, and also comprises sequential or simultaneous administration of a vaccine-enhancing agent (e.g., losartan), which is given at or around the time of vaccination and reduces recruitment of immune suppressive myeloid cells.

In some embodiments, a method for vaccinating a subject comprises administering an inactivated microbial vaccine composition comprising inactivated microbes to a subject in need thereof. An adjuvant may optionally be administered at the time of vaccination. In some embodiments, an adjuvant is administered after vaccination to boost the immune response, for example about 6 hours, about 12 hours, about 24 hours, about 36 hours, about 48 hours, about 60 hours, or about 72 hours after vaccination. In some embodiments, the adjuvant comprises liposomes, e.g., cationic liposome-DNA complexes (CLDC). In some embodiments, a vaccine-enhancing agent such as losartan may be administered at or around the time of the vaccination. In some embodiments, a vaccine-enhancing agent such as losartan may be administered after vaccination, for example, about 6 hours, about 12 hours, about 24 hours, about 36 hours, about 48 hours, about 60 hours, or about 72 hours after vaccination. In some embodiments, a vaccine-enhancing agent such as losartan may be administered to the subject daily for an effective number of days, optionally beginning on the day that the vaccine is administered. In some embodiments, the vaccine-enhancing agent (e.g., losartan) is administered at a dose of between about 5 and about 100 mg/kg, for example about 5, about 10, about 15, about 20, about 25, about 30, about 35, about 40, about 45, about 50, about 55, about 60, about 65, about 70, about 75, about 80, about 85, about 90, about 95, or about 100 mg/kg, including all values and subranges that lie therebetween.

The inactivated microbial vaccine may elicit an immune response in the subject. In some embodiments, the immune response may include one or more of the following: (i) upregulation of immunoglobulin expression (e.g., IgG, IgM), (ii) T-cell activation, (iii) modulation of innate immune cells (e.g., myeloid cells), and (iv) revival of “exhausted” T-cell populations.

### Methods for Treating and/or Preventing a Mycobacterial Infection

Also provided herein are methods for treating and/or preventing a mycobacterial infection in a subject in need thereof. In some embodiments, the methods comprise administering to the subject an effective amount of one or more vaccine compositions described herein (e.g., an inactivated microbial vaccine). The subject may be, for example, a mammal such as a human. In some embodiments, the vaccine is administered intramuscularly. In some embodiments, the vaccine is administered subcutaneously.

In some embodiments, the methods comprise administering a booster dose of the vaccine composition. The booster dose may be administered about 1 week, about 2 weeks, about 3 weeks, about 4 weeks, about 2 months, about 3 months, about 4 months, about 5 months, about 6 months, or about 1 year after administering the vaccine composition.

Also provided is a method for treating and/or preventing a mycobacterial infection (e.g., tuberculosis) in a subject in need thereof, the method comprising administering to the subject a first vaccine composition comprising an effective amount of an inactivated microbial vaccine composition described herein; and a second vaccine composition, comprising an effective amount of the inactivated microbial vaccine composition. In some embodiments, the number of microbial cells in the first vaccine composition is greater than the number of microbial cells in the second vaccine composition. In some embodiments, the number of microbial cells in the first vaccine composition is less than the number of microbial cells in the second vaccine composition. In some embodiments, the number of microbial cells in the first vaccine composition is about the same as the number of microbial cells in the second vaccine composition. The second vaccine composition may be administered, for example, about 1 week, about 2 weeks, about 3 weeks, about 4 weeks, about 2 months, about 3 months, about 4 months, about 5 months, about 6 months, or about 1 year after the first vaccine composition.

Also provided is a method for treating and/or preventing a mycobacterial infection (e.g., tuberculosis) in a subject in need thereof, the method comprising administering to the subject a first vaccine composition comprising an effective amount of attenuated, live Mycobacterium bovis, and a second vaccine composition comprising an effective amount of an inactivated microbial vaccine composition described herein (e.g., a vaccine composition comprising inactivated M. tuberculosis). In some embodiments, the first vaccine composition comprises the BCG vaccine. In some embodiments, the number of microbial cells in the first vaccine composition is greater than the number of microbial cells in the second vaccine composition. In some embodiments, the number of microbial cells in the first vaccine composition is less than the number of microbial cells in the second vaccine composition. In some embodiments, the number of microbial cells in the first vaccine composition is about the same as the number of microbial cells in the second vaccine composition. In some embodiments, the second vaccine composition is administered about 1 week, about 2 weeks, about 3 weeks, about 4 weeks, about 2 months, about 3 months, about 4 months, about 5 months, about 6 months, or about 1 year after the first vaccine composition. In some embodiments, the first vaccine composition is administered subcutaneously or intramuscularly. In some embodiments, the second vaccine composition is administered subcutaneously or intramuscularly. For example, in some embodiments, the first vaccine composition and the second vaccine composition are each administered subcutaneously. In some embodiments, the first vaccine composition and the second vaccine composition are each administered intramuscularly. In some embodiments, the first vaccine composition is administered subcutaneously, and the second vaccine composition is intramuscularly. In some embodiments, the first vaccine composition is administered intramuscularly, and the second vaccine composition is administered subcutaneously.

### Use of Inactivated Microbes as Immunogens

Microbes inactivated according to the methods disclosed herein, and compositions comprising the same, may be used as immunogens. An immunogen is a molecule capable of eliciting an immune response in a subject. For example, an immunogen may be used to stimulate a subject's immune system to produce antibodies against a microbe (e.g., Mycobacterium tuberculosis). Because an intact microbe is used, the subject's immune system will produce antibodies directed against a wide array of microbial antigens (i.e., polyclonal antibodies). The antibodies can then be harvested and purified and can be used therapeutically to treat subjects with a disease (e.g., tuberculosis). The subject may be, for example, a mammal or an avian, as described above. In some embodiments, the subject is a chicken, a cow, or a horse. In some embodiments, the subject is a human.

In some embodiments, an inactivated microbe (e.g., Mycobacterium tuberculosis), or a composition comprising the same, is administered to a subject in order to elicit immune response in the subject. The immune response may comprise, for example, production of antibodies that bind to and/or neutralize the microbe (e.g., Mycobacterium tuberculosis).

In some embodiments, a method of producing antibodies against a microbe (e.g., Mycobacterium tuberculosis) comprises administering to a subject an inactivated microbe or a composition comprising the same. In some embodiments, the method comprises recovering the antibodies from the subject, for example by obtaining a blood sample from the subject. In some embodiments, the method comprises recovering an antibody-producing cell from the subject, for example by obtaining one or more splenic cells from the subject.

In some embodiments, a method of treating a first subject in need thereof comprises administering to the first subject antibodies obtained from a second subject, wherein the second subject was immunized with inactivated microbes (e.g., Mycobacterium tuberculosis). In some embodiments, a method of treating a first subject in need thereof comprises administering to the first subject antibodies produced by cells from a second subject, wherein the second subject was immunized with inactivated microbes (e.g., Mycobacterium tuberculosis).

The compositions described herein, including the immunogenic compositions, can be used to provoke an immune response in a host. In some embodiments, a composition comprising an inactivated microbe is administered to a host, in order to provoke an immune response in the host. The immune response may comprise, for example, production of antibodies against various different antigens on the inactivated microbe.

As used herein, the term “polyclonal antibodies” refers to antibodies that are secreted by different B cell lineages. In contrast, a monoclonal antibody comes from a single-cell lineage. A polyclonal antibody comprises a collection of immunoglobulin molecules that react against a specific antigen, each identifying a different epitope. Typically, when a microbe is administered to a host, the host produces a polyclonal antibody against the microbe that was administered. The polyclonal antibody will typically include a mixture of antibodies produced by different B cell lineages, and each antibody may identify a different epitope.

In some embodiments, a method of producing a polyclonal antibody that binds to a microbe comprises i) generating an inactivated microbe by contacting the microbe with a dose of UV light in the presence of riboflavin; ii) administering the inactivated microbe to a host, wherein the host produces a polyclonal antibody; and iii) recovering the polyclonal antibody. In some embodiments, the microbe is M. tuberculosis.

In some embodiments, a method of producing a polyclonal antibody that binds to a microbe (e.g., M. tuberculosis ) comprises i) generating an inactivated microbe by contacting the microbe with a dose of UV light in the presence of riboflavin; ii) administering the inactivated microbe to a host, wherein the host produces a polyclonal antibody; and iii) recovering the polyclonal antibody.

The inactivated microbe may be administered to the host by standard methods used in the art. For example, the inactivated microbe may be administered intramuscularly, intramucosally, intranasally, subcutaneously, intratumorally, intradermally, transdermally, intravaginally, intraperitoneally, intrarectally, intra-articularly or intra-lymphatically, orally or intravenously.

In some embodiments, the methods comprise administering an adjuvant to the host. Acceptable adjuvants are listed above, including adjuvants that promote a Th1-type response.

In some embodiments, the host is a mammal. In some embodiments, the host is a non-human primate, a bovine, an ovine, a caprine, an equine, a feline, a canine, a rodent or a lagomorph. The rodent may be, for example, a mouse, a rat, a guinea pig, or a hamster. In some embodiments, the host is a human. In some embodiments, the host is an avian. In some embodiments, the host is a chicken, a duck, a goose, a quail, a turkey, a pheasant, a parrot, or a parakeet.

The polyclonal antibody may be recovered from a host in numerous different ways. For example, a blood sample (e.g., whole blood, serum, or plasma) containing the polyclonal antibody may be recovered from the host. In some embodiments, the antibody may be recovered from an immune cell producing an antibody (e.g., a B-cell) that is obtained from the host. In some embodiments, the antibody may be recovered from biological material produced by the host. For example, antibodies may be recovered from an egg (e.g., a chicken egg) produced by the host. In some embodiments, the antibodies may be isolated or purified after they are recovered from the host. In some embodiments, the antibodies are not isolated or purified after they are recovered from the host.

In some embodiments, the host is a chicken, and the polyclonal antibody is recovered from an egg produced by the host. In some embodiments, the polyclonal antibody is recovered from the blood of the host. In some embodiments, the polyclonal antibody is recovered from B cells of the host.

In some embodiments, about 1×10⁵ to about 1×10⁸ inactivated microbes are administered to a host in each administration. For example, about 1×10⁵, about 5×10⁵, about 1×10⁶, about 5×10⁶, about 1×10⁷, about 5×10⁷, or about 1×10⁸ inactivated microbes are administered to a host per administration. In some embodiments, the administered dose is a split dose, wherein the total number of microbes for administration is divided into 2, 3, 4, 5, 6, 7, 8, 9, or 10 sub-doses.

One or more sub-dose may be administered to the host peripherally, at different locations on the host's body. Each sub-dose may be administered at approximately the same time, or administration of the sub-doses may be staggered. For example, sub-doses may be administered at intervals of 15 minutes, 20 minutes, 30 minutes, 45 minutes, 1 hour, or 3 hours.

In some embodiments, the vaccine composition may be administered once, or more than once to the host. In some embodiments, the vaccine is administered once, twice, three times, four times, five times, six times, seven times, eight times, nine times, or ten times to the host.

The composition may be administered to the host every day, about every 3 days, about every 7 days, about every fourteen days, about once per month, or about once per year. In some embodiments, the composition is administered at least once per week, at least every two weeks, or at least once every six months. In some embodiments, the composition is administered once, twice, three times, four times, five times, six times, seven times, eight times, nine times, ten times, twelve times, fifteen times, twenty times, or twenty-five times in a year.

In some embodiments, a first dose (e.g., a prime dose) of the composition is administered, followed by a second dose (e.g., a boost dose) to the host. In some embodiments, the second dose is administered about 1 week, about 2 weeks, about 3 weeks, about 4 weeks, about 2 months, about 3 months, about 4 months, about 5 months, about 6 months, or about 1 year after the first dose. In some embodiments, the number of microbes in the first dose is greater than the number of microbes in the second dose. For example, the number of microbes may be about 10%, about 20%, about 30%, about 40%, about 50%, about 60%, about 70%, about 80%, about 90%, or about 100% greater than the number of microbes in the first dose. In some embodiments, the number of microbes in the first dose is less than the number of microbes in the second dose. For example, the number of microbes may be about 10%, about 20%, about 30%, about 40%, about 50%, about 60%, about 70%, about 80%, about 90%, or about 100% less than the number of microbes in the first dose. In some embodiments, the number of microbes in the first dose is about the same as the number of microbes in the second dose.

In some embodiments, a first inactivated composition and a second inactivated composition are administered to the host. In some embodiments, the second inactivated composition is administered after the first inactivated composition to boost the immune response. In some embodiments, immune response in the subject is monitored between administration of the first composition and the second composition. In some embodiments, the second composition is administered when it is determined that the host has not exhibited a satisfactory immune response following administration of the first composition.

In some embodiments, a method for administering an immunogenic composition to a host comprises administering a composition comprising inactivated microbes to the host. An adjuvant may optionally be administered at the time of vaccination. In some embodiments, an adjuvant is administered after vaccination to boost the immune response, for example about 6 hours, about 12 hours, about 24 hours, about 36 hours, about 48 hours, about 60 hours, or about 72 hours after administration.

Administration of an immunogenic composition (e.g., a composition comprising inactivated microbes) may elicit an immune response in the host. In some embodiments, the immune response may include one or more of the following: (i) upregulation of immunoglobulin expression (e.g., IgG, IgM, IgA, IgE), (ii) T-cell activation, (iii) modulation of innate immune cells (e.g., myeloid cells), and (iv) revival of “exhausted” T-cell populations.

Also provided are polyclonal antibodies produced according to the methods described herein, and polyclonal antibodies produced using the immunogenic compositions described herein. The polyclonal antibodies may comprise, for example, IgG, IgM, IgA, IgY, or IgE, or mixtures thereof. In some embodiments, the polyclonal antibodies bind to a microbe or an antigen thereof. In some embodiments, the polyclonal antibodies bind to the same microbe as was used to produce the polyclonal antibody in the host.

In some embodiments, a polyclonal antibody directed against M. tuberculosis is provided. In some embodiments, the polyclonal antibody binds to an antigen on the surface of a M. tuberculosis. In some embodiments, the antigen is not substantially modified by treatment using UV light in the presence of riboflavin.

### Diagnostic Methods

Microbes inactivated according to the methods disclosed herein may be used in diagnostic compositions and methods. For example, the inactivated microbes (for example, Mycobacterium tuberculosis) may be used to detect the presence of an anti-microbial antibody (e.g., a neutralizing antibody) in a biological sample. The biological sample may be, for example, blood (e.g., whole blood, serum, or plasma), stool, urine, saliva, or swab specimens of the nostril, throat, cervix, or urethra. Because intact microbes are used, antibodies that bind to many different targets (i.e., different microbial epitopes) may be detected using a single assay.

In some embodiments, an inactivated microbe is coupled to and/or immobilized on a substrate. The substrate may be biological, nonbiological, organic, inorganic, or a combination of any of these, existing as particles, strands, precipitates, gels, sheets, tubing, spheres, containers, capillaries, pads, slices, films, plates, slides, etc. The substrate may have any convenient shape, such as a disc, square, sphere, and a circle. The substrate may, in some embodiments, form a rigid support. In some embodiments, the substrate and its surface may be chosen to provide appropriate light-absorbing characteristics. For instance, the substrate may be a polymerized Langmuir Blodgett film, functionalized glass, Si, Ge, GaAs, GaP, SiO₂, SiN₄, modified silicon, or any one of a wide variety of polymers such as (poly)tetrafluoroethylene, (poly)vinylidenedifluoride, or combinations thereof In some embodiments, the substrate may be a bead, a resin, a membrane, a fiber, a polymer, a matrix, a chip, a microplate or a tissue culture vessel.

In some embodiments, the substrate is pre-treated before the inactivated microbe is coupled thereto. For example, the substrate may be treated with an enzyme, such as a DNAse, RNAse or protease. In some embodiments, the substrate may be coated with a polymer or a carbohydrate. In some embodiments, the substrate may be coated with a protein, such as an antibody, antibody fragment (e.g. Fab), or antibody derivative (e.g., a single chain variable fragment (scFv)).

The inactivated microbe may be coupled to the substrate using various different methods. For example, the inactivated microbe may be reversibly or irreversibly coupled to the substrate. In some embodiments, the inactivated microbe is coupled to the substrate via a linker. The linker may be a chemical or a protein linker. The chemical linker may be, for example, a carbohydrate linker, a polyether linker, a fatty acid linker, or a lipid linker. In some embodiments, the protein linker may comprise about 1 to about 50 amino acids. In some embodiments, the protein linker is a flexible linker (i.e., a linker that comprises small polar amino acids, including threonine, serine, and/or glycine). In some embodiments, the protein linker is not flexible (i.e., a linker that comprises one or more proline residues).

In some embodiments, the inactivated microbes described herein may be used in a method for detecting the presence of an antibody in a biological sample. The antibody may be an antibody that binds to the microbe, such as a neutralizing antibody. In some embodiments, the method for detecting an antibody in a biological sample comprises contacting the biological sample with a microbe particle that is coupled to a substrate. In some embodiments, the antibody binds to the microbe particle that is coupled to the substrate, thereby immobilizing the antibody. In some embodiments, the method further comprises contacting the immobilized antibody with a second antibody, such as a detection antibody. The detection antibody may be coupled to, for example, a fluorophore, or to an enzyme (e.g., horseradish peroxidase (HRP)) capable of cleaving a substrate (e.g., a fluorescent substrate).

In some embodiments, the inactivated microbes described herein may be used in an ELISA-based assay. An ELISA (enzyme-linked immunosorbent assay) is a plate-based assay technique designed for detecting and quantifying proteins, such as antibodies, in a biological sample. In an ELISA, an antigen (e.g., an inactivated microbe) is typically immobilized to a solid surface and then complexed with an antibody that is linked to an enzyme. Detection may be accomplished by measuring the activity of the reporter enzyme via incubation with the appropriate substrate to produce a measurable product. There are several different types of ELISAs commonly used, including direct ELISAs, indirect ELISAs, sandwich ELISAs, and competitive ELISAs.

In some embodiments, an inactivated Mycobacterium tuberculosis is coupled to a substrate. In some embodiments, the inactivated Mycobacterium tuberculosis is contacted with a biological sample comprising an antibody that binds to the Mycobacterium tuberculosis. A complex is formed between the antibody and to the microbe particle that is coupled to the substrate, thereby immobilizing the antibody. After the biological sample (including any unbound antibodies) is washed away, the antibody is contacted with a detection antibody. The binding of the antibody to the Mycobacterium tuberculosis is measured, for example, by detecting the amount of detection antibody bound. In some embodiments wherein the detection antibody is conjugated to an enzyme (e.g., an HRP), binding of the antibody to the Mycobacterium tuberculosis may be measured by measuring the amount of a fluorophore produced when an appropriate substrate is added to the sample.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world. Further, PCT International Publication Nos. WO 2021/178877 and WO 2019/183320 are hereby incorporated by reference in their entireties.

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

Unless the context indicates otherwise, it is specifically intended that the various features described herein can be used in any combination.

It is to be understood that the description above as well as the examples that follow are intended to illustrate, and not limit, the scope of the invention. Other aspects, advantages and modifications within the scope of the invention will be apparent to those skilled in the art to which the invention pertains.

### EXAMPLES

The following examples, which are included herein for illustration purposes only, are not intended to be limiting.

### Example 1: Inactivation of Mycobacterium smegmatis Using Riboflavin and UV Light

Mycobacterium smegmatis is a Biosafety Level-1 (BSL-1) environmental microbe, which was used in this experiment due to its similarity to Mycobacterium tuberculosis. Inactivation of Mycobacterium smegmatis was performed using riboflavin and UV light in a Mirasol° device (Terumo BCT, Lakewood, CO). While Mycobacteria spp. primarily use homologous recombination (HR) and non-homologous end joining (NHEJ) to repair double-stranded breaks in chromosomal DNA, M. smegmatis can also use a single strand annealing (SSA) pathway that operates independently of RecA. Without being bound by any theory, it is believed that because M. smegmatis has additional protection against double-stranded breaks, and damage to DNA repair machinery, it presents a more significant challenge for inactivation as compared to pathogenic Mycobacterium species.

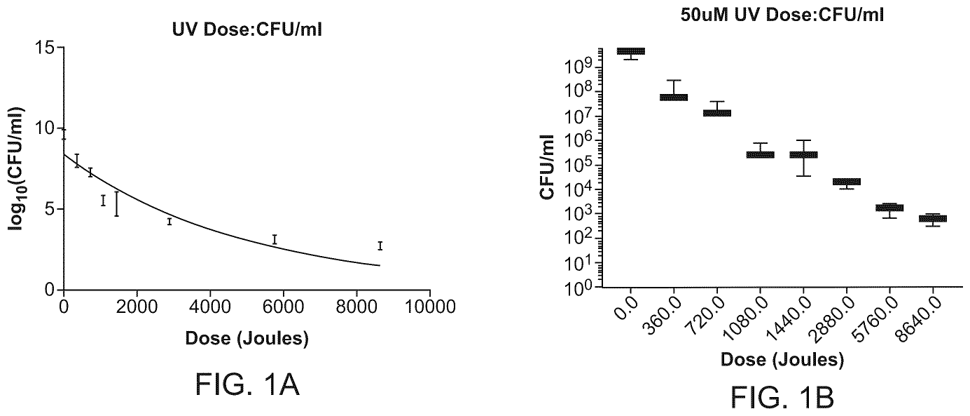

7H11 agar plates were inoculated with 1 ml M. smegmatis stock suspension and growth was observed over a period of 3-4 days. The lawn of colonies was transferred to a Fernbach flask filled with 1 L gas media. After 3 days incubation at 37° C., cells were concentrated into a pellet. The pellets were exposed to increasing doses of UV light in 300 mL of solution comprising 50 μM riboflavin. The container utilized for treatment was a standard Mirasol® PRT illumination bag (citrate plasticized PVC, 1 Liter volume, Terumo BCT, Lakewood, CO). Energy dose utilized was measured on the device with a calibrated optical meter. Dose response curves for M. smegmatis cells treated with different doses of UV light are shown in FIG. 1.

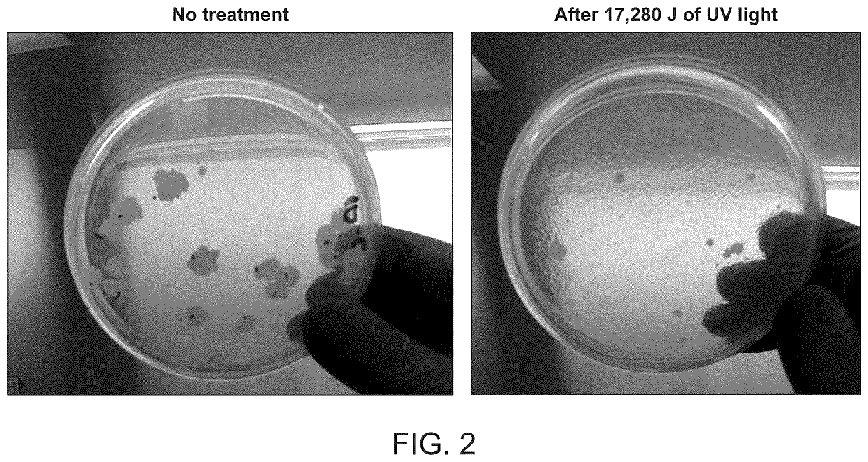

FIG. 1A shows the reduction in the colony forming units (CFUs/mL), with increasing dose of the UV light in the presence of riboflavin. The reduction in CFU is biphasic, with an initial phase of faster inactivation spanning an energy dose range of 0-2000 J, followed by a slower inactivation phase spanning an energy dose range of 2000 J-8000 J. Without being bound by any theory, it is believed that the initial phase might reflect the inactivation of single cells in solution, while the latter phase might reflect the slower inactivation of cells in clumps. The dose response curve predicts that 1 CFU/ml will be obtained at about 9,676 J of UV light, and 0.1 CFU/ml will be obtained at about 22,100 J. FIG. 1B shows that while about 109 CFUs/mL are obtained after control treatment with no UV light, only about 103 CFUs/mL are obtained after treatment with 8640 J of UV light in the presence of riboflavin. At even higher doses of UV light (>17,200 J) in the presence of riboflavin, a mere 12 CFUs/mL are obtained (see FIG. 2).

The results show that M. smegmatis can be inactivated by exposure to UV light in the presence of riboflavin, as indicated by the reduction in the number of viable bacteria after the treatment.

### Example 2: Inactivation of Mycobacterium tuberculosis Using Riboflavin and UV Light

As described in Example 1, M. tuberculosis H37Rv cells were treated with different doses of UV light (as indicated in Table 1) in 300 mL of solution comprising 50 μM riboflavin. Microscope examination revealed that the preparations contained clumps of cells, making it difficult to count the number thereof. The samples were then recounted using the Petroff-Hausser counter after sonication for 3 min and vortexing for 30 sec (2 times) so that the correct number of cells could be added for culturing in Mycobacteria Growth Indicator Tube (MGIT). The samples exposed to 8640 Joules were at a concentration of 1.46×106. The samples exposed to 17280 Joules were at 2.83×10⁶.

MGIT was used for the detection of mycobacteria. The BACTEC™ MGIT™ automated mycobacterial detection system utilizes modified Middlebrook 7H9 Broth base with OADC enrichment and PANTA antibiotic mixture (polymyxin B, amphotericin B, nalidixic acid, trimethoprim and azlocillin) in a Mycobacteria Growth Indicator Tube (MGIT). A fluorescent compound is embedded in silicone on the bottom of each of the tubes, which is sensitive to the presence of oxygen dissolved in the broth. Initially, the large amount of dissolved oxygen quenches the emissions from the compound and little fluorescence can be detected. Later, actively respiring M. tuberculosis cells consume the oxygen, and a detectable fluorescent signal is produced. The BACTEC™ MGIT™ automated mycobacterial detection system monitors the tubes for increasing fluorescence. Analysis of the fluorescence was used to determine if the tube is instrument-positive; i.e., the test sample contains viable organisms. Culture tubes which showed no fluorescence were monitored for a minimum of 42 days. The time taken for a sample to show fluorescence was measured as the “time to positive” value.

TABLE 1
MGIT Assay Results

| | | | UV | Time to |
|---|---|---|---|---|
| | | Sonicated? | light | positive |
| Sample | | (Yes/ No) | dosage | (hours) |
| H37Rv- | Replicate 1 | No | NA | 72 |
| Untreated | Replicate 2 | No | NA | 73 |
| H37Rv- | Replicate 1 | No | 8640 J | 436* |
| 8640 J | Replicate 2 | No | 8640 J | 0 |
| H37Rv- | Replicate 1 | No | 17280 J | 0 |
| 17280 J | Replicate 2 | No | 17280 J | 0 |
| H37Rv- | Replicate 1 | Yes | NA | 79 |
| Untreated | Replicate 2 | Yes | NA | 74 |
| H37Rv- | Replicate 1 | Yes | 8640 J | 0 |
| 8640 J | Replicate 2 | Yes | 8640 J | 0 |
| H37Rv- | Replicate 1 | Yes | 17280 J | 0 |
| 17280 J | Replicate 2 | Yes | 17280 J | 0 |

*This fluorescence is likely due to a contaminant.

As shown in Table 1, while untreated H37Rv cells were viable and actively proliferating, as indicated by a fluorescence signal detected in about 72-79 hours, H37Rv cells treated with UV light (either 8640 J or 17280 J) in the presence of riboflavin did not give a fluorescence signal even after prolonged incubation. The fluorescence positive signal seen in the replicate 1 sample of “H37Rv-8640 J” is thought to be due to a contamination. To confirm this, an aliquot was taken from the MGIT and streaked onto 7H11 agar. A colony was observed on the 7H11 within approximately 3 days. The colony was streaked onto a glass slide for acid-fast and Gram staining. The stains showed that the organism was acid-fast and gram variable. This sample was therefore disregarded. These results confirmed that the disclosed methods can be used reproducibly and effectively to stop proliferation of M. tuberculosis cells.

In addition, the viability of H37Rv cells treated with UV light in the presence of riboflavin was also assayed using alamarBlue® assay, which uses a cell viability assay reagent containing a cell permeable, non-toxic and weakly fluorescent blue indicator dye called resazurin. The assay involved adding alamarBlue® reagent at 10% volume of the culture in a well, and incubating at 37° C. for about 1-4 hours at a pH range of about 6.8 to about 7.4, followed by fluorescence measurement at 570 nm excitation wavelength and 600 nm emission wavelength. The results from the assay, shown in FIG. 3, support the MGIT results described above. H37Rv cells treated with either 8640 J or 17280 J UV light in the presence of riboflavin showed no fluorescence and were indistinguishable from the negative control, while the positive control showed robust fluorescence that increased with time. The negative control uses media in which there are no tuberculosis bacteria present (i.e., it is the media in which the cells are grown). The positive control is a tuberculosis-containing sample which has been grown up and is at the same concentration as the inactivated material, but has had no riboflavin and no UV light exposure. These results further demonstrate that the disclosed methods can be used to inactivate M. tuberculosis cells.

### Example 3: Assessment of Immune Responses Induced by Inactivated M. tuberculosis H37Rv Cells

Vaccine compositions comprising M. tuberculosis H37Rv cells inactivated by the methods disclosed herein were administered to mice to examine whether a robust immune response is generated. While tuberculosis is not fatal in mice, an immune response is still seen, allowing evaluation of the immune response elicited by the inactivated M. tuberculosis cells to a mammal.

Based on the data provided in Example 2, the inactivated M. tuberculosis H37Rv 17280 J sample was used as a vaccine. After the samples had been confirmed to contain no viable M. tuberculosis, C57BL/6 mice (5 per group; approximately 8 to 10 weeks-old, Jackson Laboratory) were acclimated for 2 weeks. Mice were placed into groups of five, with Group 1 receiving 100 uL sterile, pyrogen-free saline subcutaneously. Group 2 was vaccinated with 5×10⁴ CFU BCG Pasteur via the subcutaneous route. Group 3 was vaccinated subcutaneously with approximately 1×10⁶ 17280 J. M. tuberculosis H37Rv, and Group 4 was vaccinated subcutaneously with approximately 1×10⁵ 17280 J. M. tuberculosis H37Rv. As further controls, Group 5 was vaccinated subcutaneously with 1×10⁶ g-irradiated M. tuberculosis H37Rv, and Group 6 was vaccinated subcutaneously with 1×10⁶ g-irradiated M. tuberculosis H37Rv (Inactivated by exposure to 2.4 mRads of ionizing g-irradiated using a 137 Cs source). Confirmation of inactivation was by plating on 7H11 agar and inoculation into BACTEC™ (BD Biosciences) tubes and incubating in a MGIT320® for up to 42 days to assess time to positivity. Vaccination groups are summarized in Table 2.

TABLE 2
Vaccination Groups

| Treatment | | No. of |
|---|---|---|
| Group No. | Vaccine Composition | Mice |
| 1 | Saline | 5 |
| 2 | BCG Pasteur | 5 |
| 3 | 17280J. M. tuberculosis H37Rv 10⁶ | 5 |
| | bacteria | |
| 4 | 17280J. M. tuberculosis H37Rv 10⁵ | 5 |
| | bacteria | |
| 5 | Irradiate M. tuberculosis H37Rv 10⁶ | 5 |
| | bacteria | |
| 6 | Irradiate M. tuberculosis H37Rv 10⁶ | 5 |
| | bacteria | |

After inoculation, mice were rested for 30 days. Mice were then infected with a low dose aerosol of virulent M. tuberculosis H37Rv, that deposited approximately 100 colony forming units (CFU) in the lung. A separate group of mice were used to determine the day 0 infection CFU. At day 30 post-infection, mice were euthanized to determine mycobacterial burden, extent of pulmonary pathology and immunity.

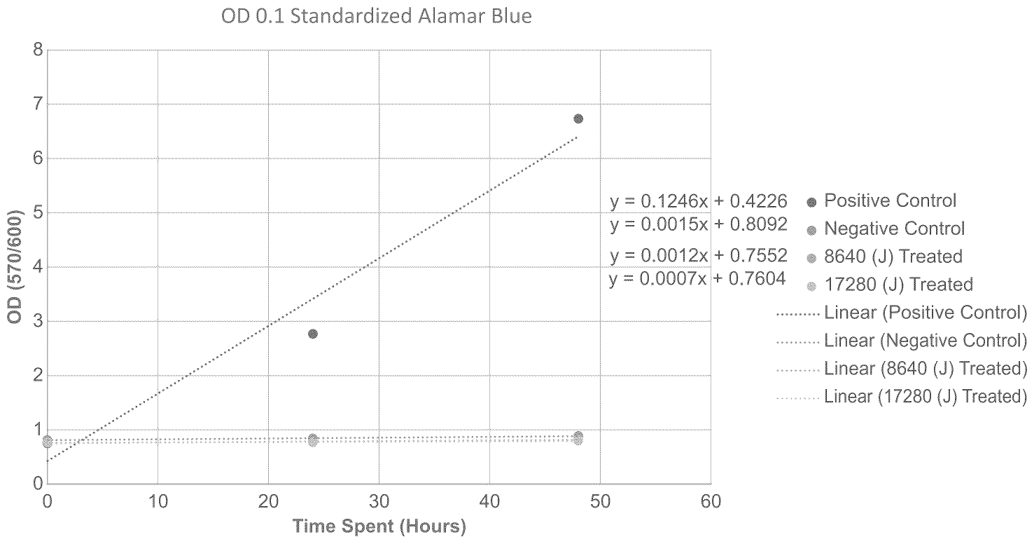

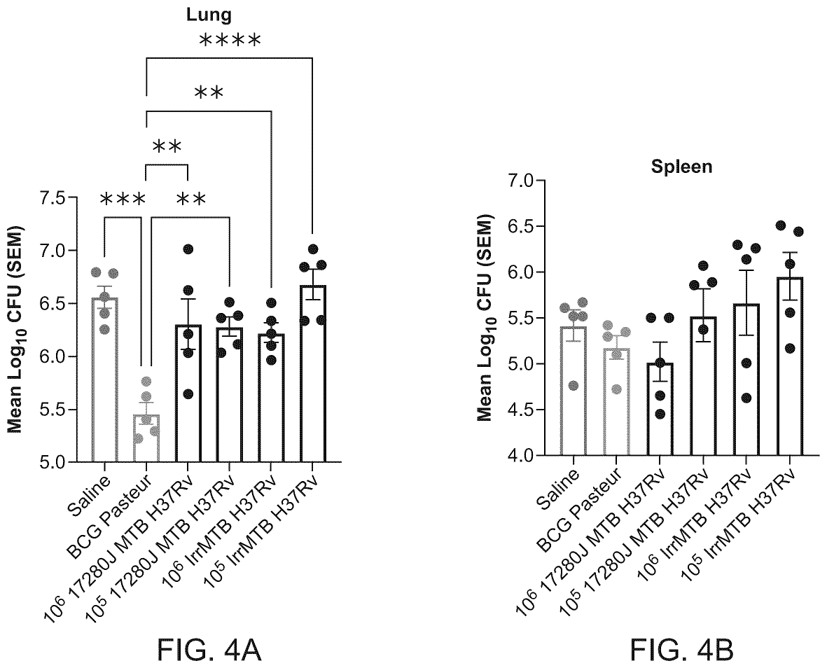

Lung and spleen were excised, and results of post-infection analysis thereof are shown in Table 3, below, and in FIG. 4A and FIG. 4B. In Table 3, Mean Log₁₀ CFU Reduction was calculated by subtracting the mean Log₁₀ CFU for the treatment group from the Log₁₀ CFU for the saline-treated group. P value was determined using the one-way analysis of variance (ANOVA), with Tukey post-test, two-tailed unpaired tests will be used for statistical comparison between groups, using Log₁₀ transformed data. Only comparison with the saline-treated group is shown.

TABLE 3
Post-Infection Readouts in Lung and Spleen

| | Lung | | | | Spleen | | | |
|---|---|---|---|---|---|---|---|---|
| | | | Mean | | | | Mean | |
| | Mean | | Log₁₀ | | Mean | | Log₁₀ | |
| | Log₁₀ | | CFU | | Log₁₀ | | CFU | |
| Group | CFU | Std | Reduction | P value | CFU | Std | Reduction | P value |
| 1 | 6.56 | 0.23 | N/A | N/A | 5.41 | 0.37 | N/A | 0.9823 |
| 2 | 5.46 | 0.23 | 1.10 | 0.0001 | 5.20 | 0.30 | 0.22 | 0.8647 |
| 3 | 6.31 | 0.53 | 0.25 | 0.7822 | 5.02 | 0.48 | 0.39 | 0.9994 |
| 4 | 6.28 | 0.20 | 0.28 | 0.7135 | 5.53 | 0.65 | −0.12 | 0.9773 |
| 5 | 6.22 | 0.21 | 0.34 | 0.5329 | 5.67 | 0.79 | −0.25 | 0.6426 |
| 6 | 6.68 | 0.32 | −0.12 | 0.9889 | 5.95 | 0.58 | −0.54 | 0.9975 |

std = standard deviation N/A = not applicable.

A portion of the lobe of the lung was utilized to analyze pathological lesions. The excised lobe was inflated with formalin and placed in total into formalin. For processing, the lung lobe was embedded in paraffin and sections cut and stained with hematoxylin and eosin. A Pathologist examined the sections, without prior knowledge of the groups, and provided a score based on the extent of lung involvement, fibrosis and lesion type. The scoring system used is shown below in Table 4.

TABLE 4
Post-infection pulmonary pathology in the mouse

| Score | Description |
|---|---|
| 0 | No apparent changes |
| 1 | Minimal changes |
| 2 | Mild changes |
| 3 | Moderate changes |
| 4 | Marked changes |
| 5 | Severe changes |

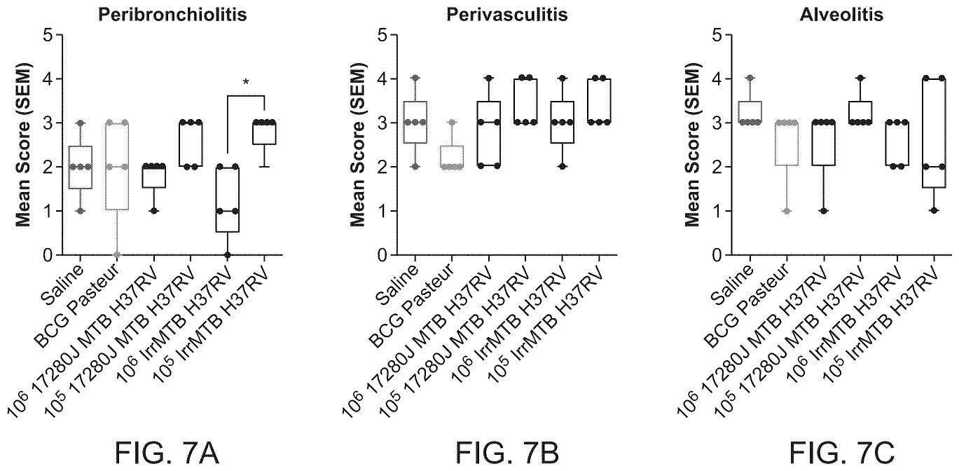

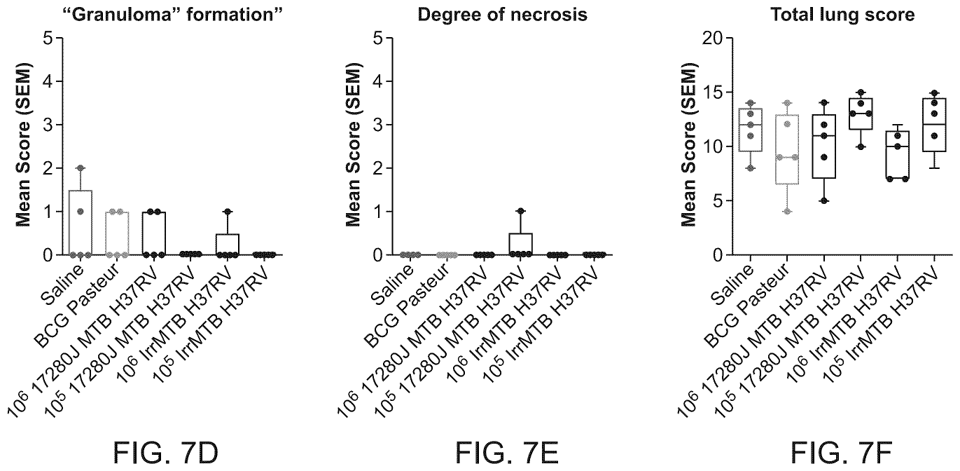

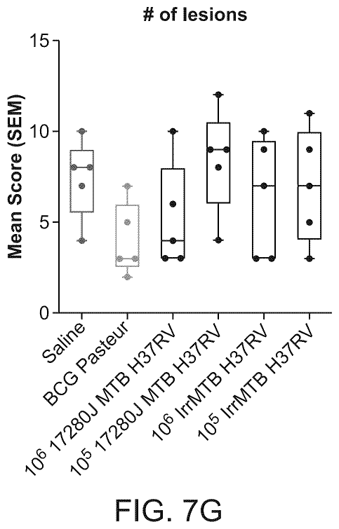

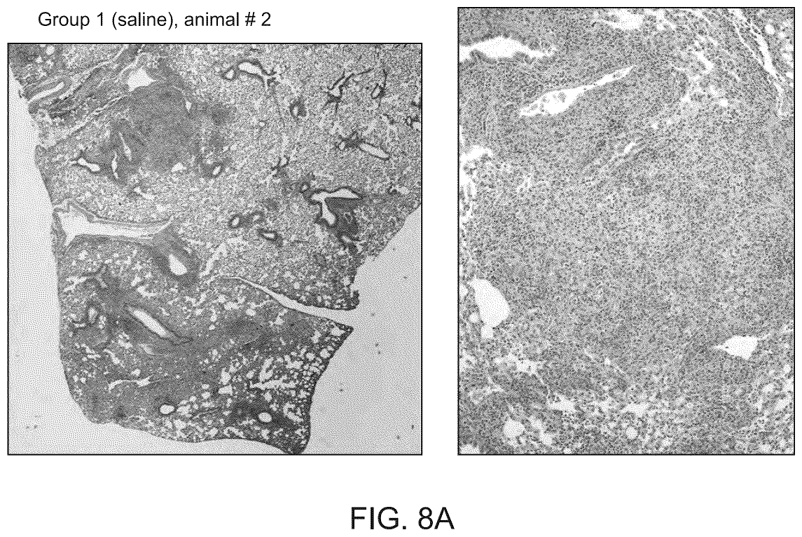

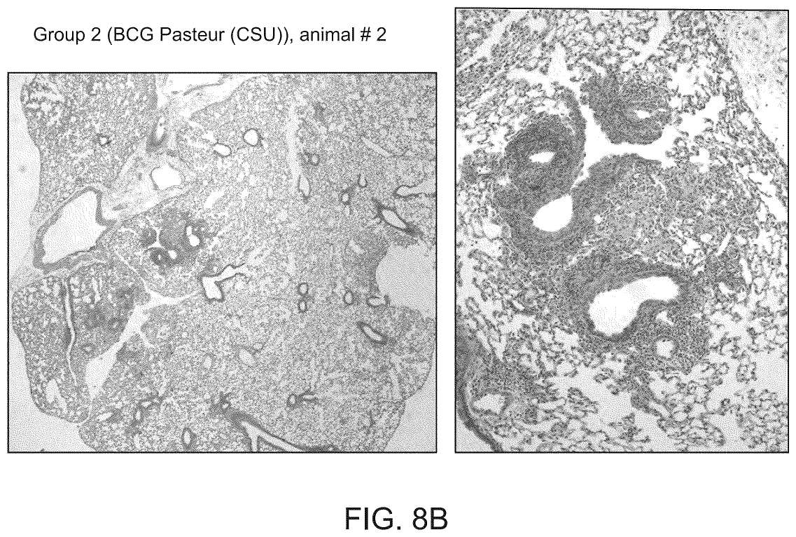

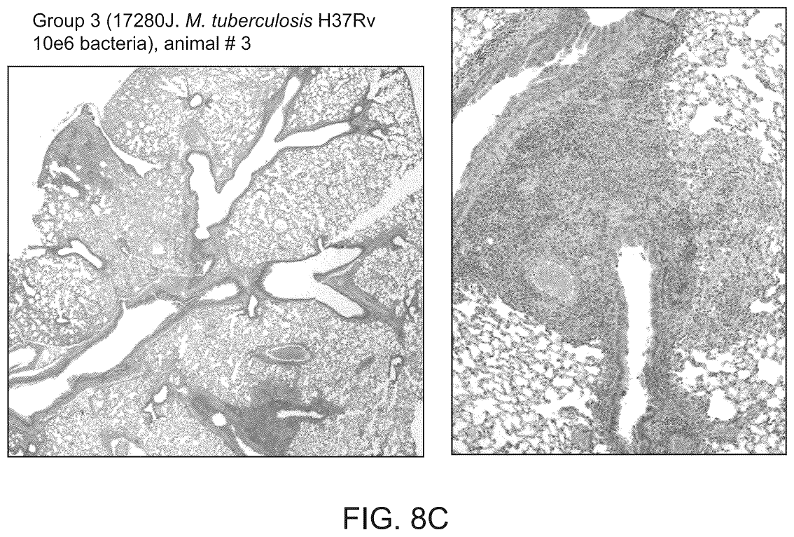

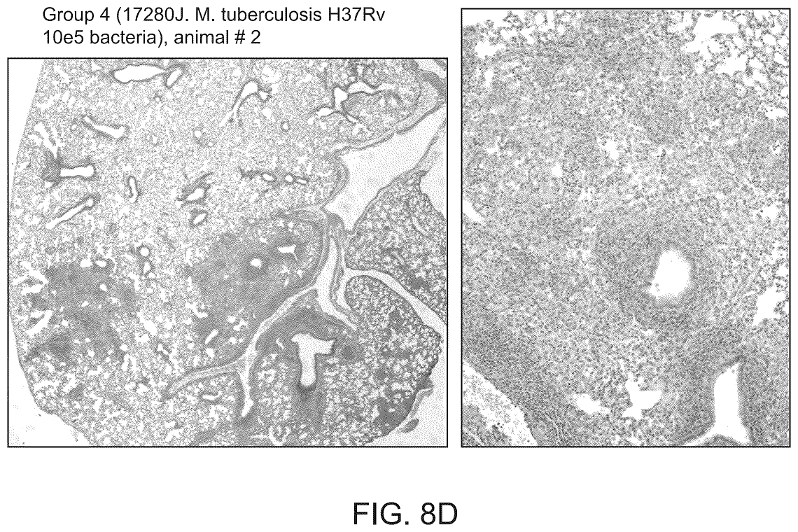

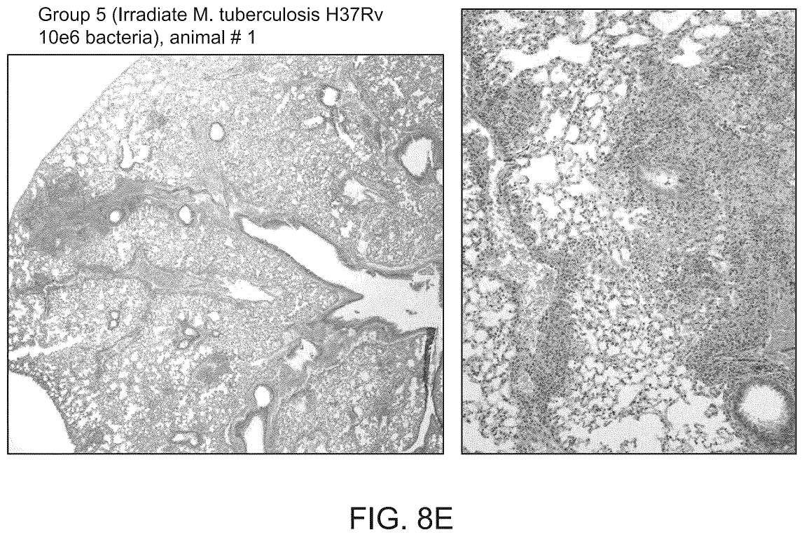

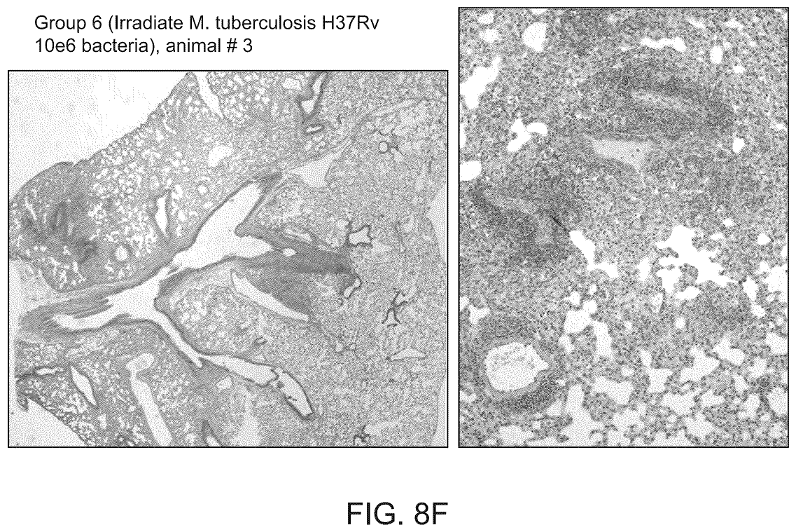

Factors that were examined included peribronchiolitis, perivasculitis, alveolitis “Granuloma” formation and degree of necrosis, to give a total lung score for lungs from each mouse. Results from this analysis are summarized in FIG. 7A-7G. Representative histology images are shown in FIG. 8A-8F.

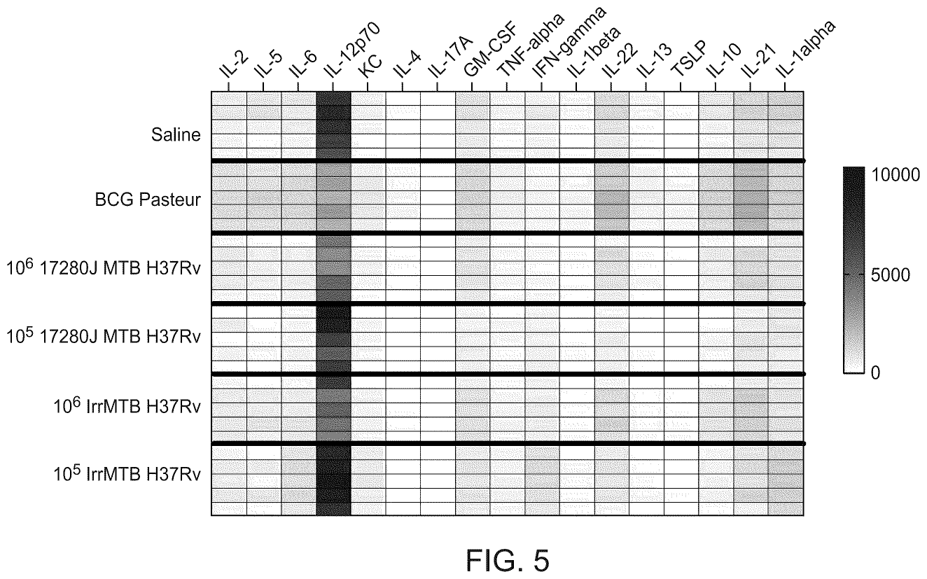

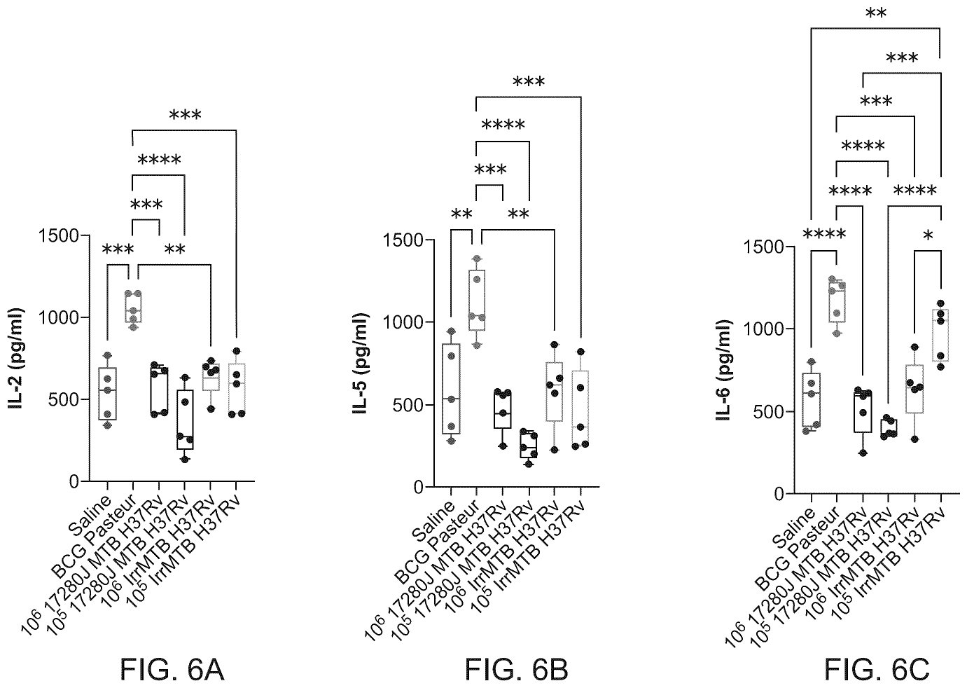

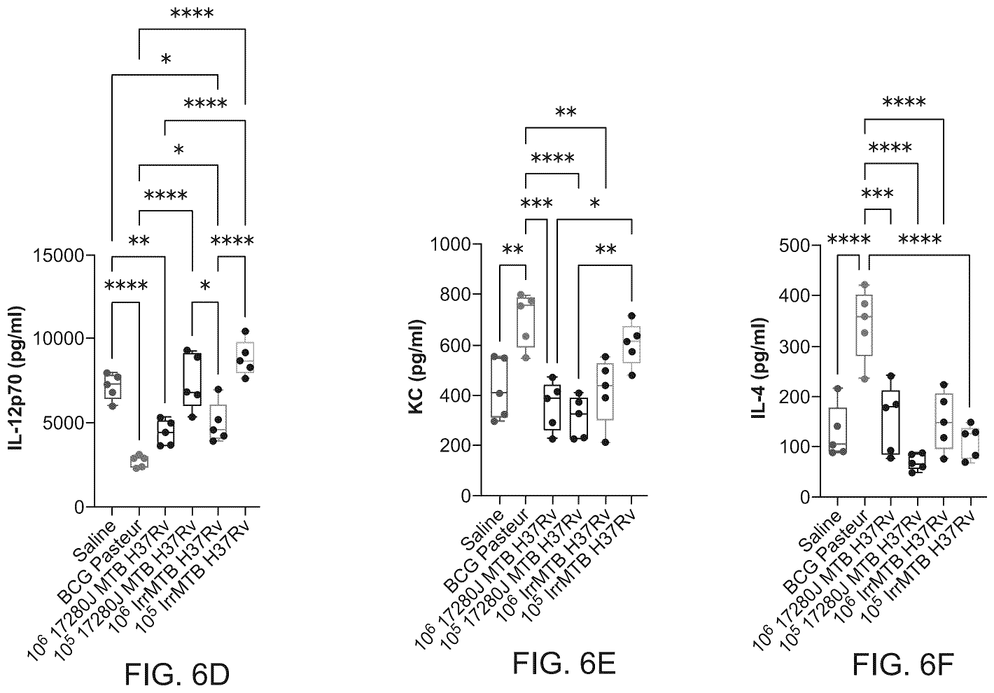

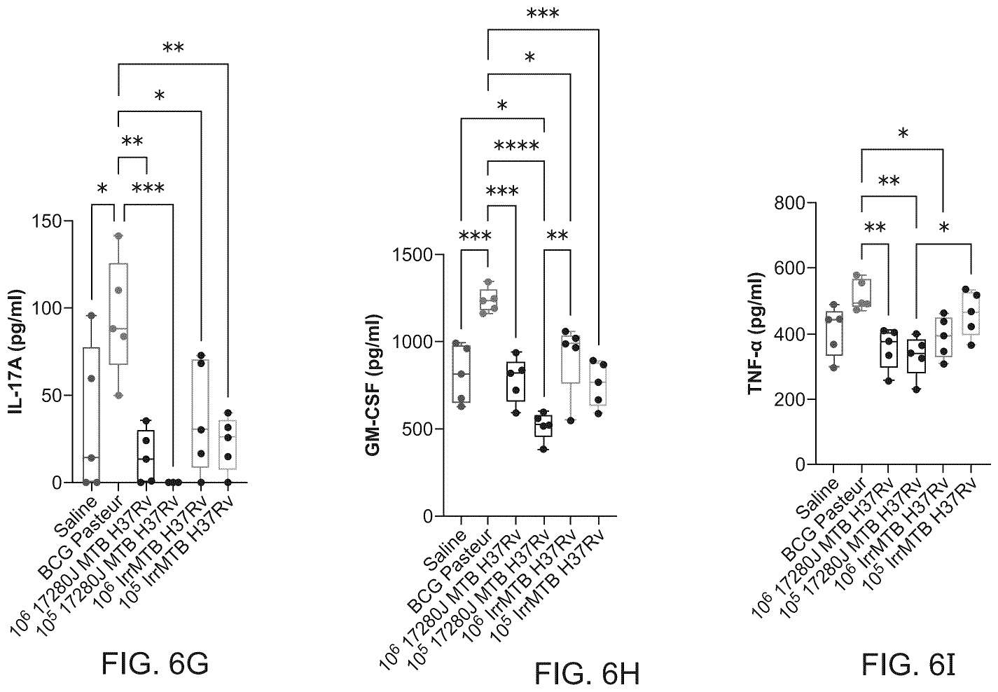

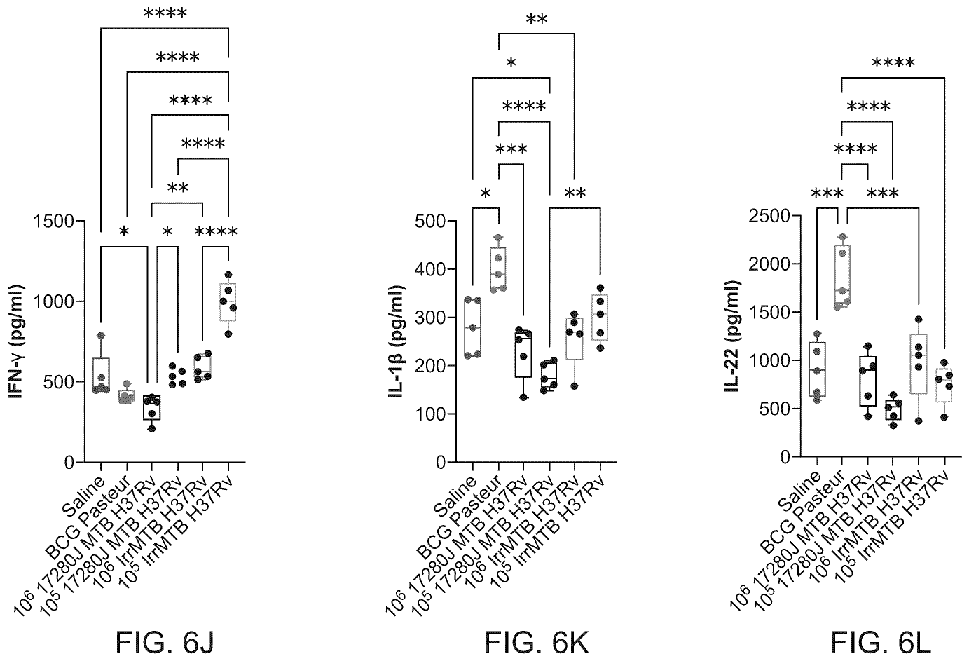

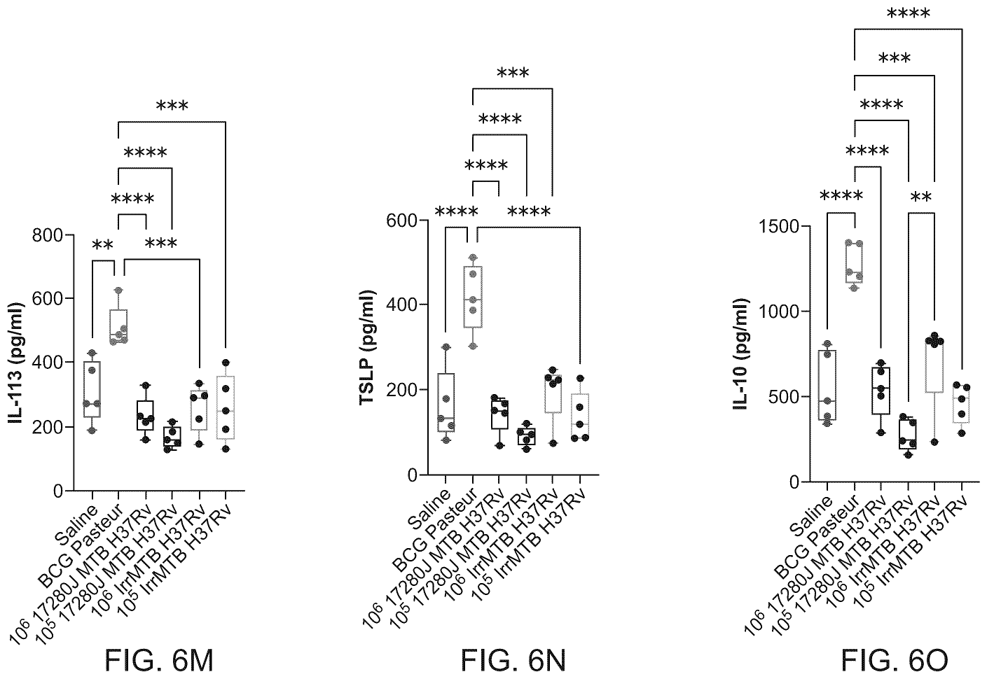

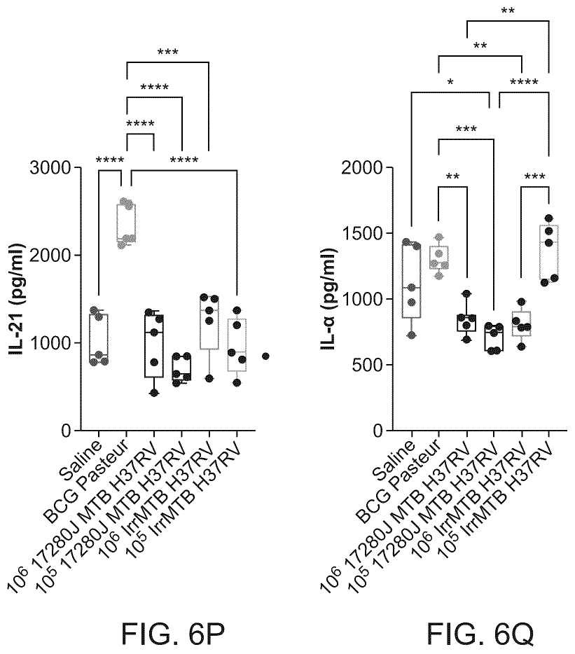

The remainder of the lung and the entire spleen were homogenized and used to assess CFU numbers. Colonies were enumerated after 16-21 days of incubation at 37° C. Lung homogenates were stored at −80° C. and used to determine the cytokine concentration using the Mouse Th1/Th2/Th17 17-Plex Panel (GM-CSF, IFN-g, IL-1a, IL-1b, IL-2, IL-4, IL-5, IL-6, IL-10, IL-12p70, IL-13, IL-17A, IL-21, IL-22, KC, TNF-a, TSLP) (Aimplex Biosciences Inc, Pomona, CA). An immunological readout of post-infection cytokines expressed in lung is provided in FIG. 5 and in FIG. 6A-6Q.

Taken together, this data demonstrates that in a mouse model, the inactivated M. tuberculosis H37Rv formulation did not provide significant reduction in CFU when compared to the saline-treated group. However, the inactivated M. tuberculosis reduced the spread of the bacteria to the spleen (i.e., log₁₀ reduction for BCG was 0.22 vs. 0.39 for inactivated M. tuberculosis). This was distinct from the changes observed with the lower dose of the inactivated M. tuberculosis material and gamma irradiated H37Rv TB isolate. With regard to production of key cytokines during infection, the 17280J inactivated M. tuberculosis induced significantly greater levels of IL-12p70, when compared to BCG vaccinated mice, suggesting the induction of Th1 immunity. There were also significantly lower concentrations of IL-10 in the 17280 J inactivated M. tuberculosis inoculated group compared to BCG vaccinated mice. Accordingly, this data indicates that it is possible to enhance the TH1 immune response using the inactivated M. tuberculosis vaccine.

### NUMBERED EMBODIMENTS

The following numbered embodiments also form part of the instant disclosure.
1. A method for inactivating a microbe, the method comprising contacting the microbe with a dose of UV light in the presence of riboflavin, wherein the microbe belongs to the genus Mycobacterium.
2. The method of embodiment 1, wherein the microbe is capable of causing disease in a subject.
3. The method of embodiment 2, wherein the subject is a human.
4. The method of any one of embodiments 1-3, wherein the microbe belongs to the Mycobacterium tuberculosis complex.
5. The method of any one of embodiments 1-4, wherein the microbe is selected from the group consisting of Mycobacterium tuberculosis, Mycobacterium africanum, Mycobacterium orygis, Mycobacterium bovis, Mycobacterium microti, Mycobacterium canetti, Mycobacterium caprae, Mycobacterium pinnipedii, Mycobacterium suricattae, and Mycobacterium mungi.
6. The method of embodiment 5, wherein the microbe is Mycobacterium tuberculosis.
7. The method of embodiment 6, wherein the microbe is Mycobacterium tuberculosis strain H37Rv.
8. The method of any one of embodiments 1-7, wherein the microbe is resistant to an antibiotic.
9. The method of embodiment 8, wherein the antibiotic is penicillin, isoniazid, clarithromycin, fluoroquinolone, amikacin, kanamycin, capreomycin, or rifamycin.
10. The method of any one of embodiments 1-9, wherein the dose of UV light is about 100 Joules to about 25,000 Joules.
11. The method of any one of embodiments 1-10, wherein the dose of UV light is about 17,000 Joules.
12. The method of any one of embodiments 1-11, wherein the method comprises altering the genome of the microbe.
13. The method of embodiment 12, wherein the method comprises selectively oxidizing one or more guanine bases in a nucleic acid of the microbe.
14. The method of embodiment 13, wherein the nucleic acid of the microbe is a DNA or an RNA.
15. The method of any one of embodiments 1-14, wherein the method does not comprise substantially altering the structure of antigens on the surface of the microbe.
16. The method of any one of embodiments 1-15, wherein the inactivated microbe is not capable of replicating.
17. The method of any one of embodiments 1-16, wherein the inactivated microbe is not capable of causing disease in a subject.
18. A vaccine composition, comprising a microbe inactivated according to any one of embodiments 1-17.
19. The vaccine composition of embodiment 18, wherein the composition comprises about 10⁴ microbial cells to about 10⁶ microbial cells.
20. The vaccine composition of embodiment 19, wherein the composition comprises about 10⁵ microbial cells.
21. The vaccine composition of any one of embodiments 18-20, wherein the composition comprises an adjuvant.
22. The vaccine composition of any one of embodiments 18-21, wherein the composition comprises a pharmaceutically acceptable carrier or excipient.
23. A vaccine composition, comprising an inactivated Mycobacterium tuberculosis, wherein the Mycobacterium tuberculosis genome comprises one or more oxidized guanine residues.
24. The vaccine composition of embodiment 23, wherein the Mycobacterium tuberculosis is Mycobacterium tuberculosis strain H37Rv.
25. The vaccine composition of embodiment 23 or 24, wherein the antigens present on the surface of the inactivated Mycobacterium tuberculosis microbe are substantially identical to those on the surface of a Mycobacterium tuberculosis microbe that has not been inactivated.
26. The vaccine composition of any one of embodiments 23-25, wherein the composition comprises about 10⁴ microbial cells to about 10⁶ microbial cells.
27. The vaccine composition of embodiment 26, wherein the composition comprises about 10⁵ microbial cells.
28. The vaccine composition of any one of embodiments 23-27, wherein the composition comprises an adjuvant.
29. The vaccine composition of any one of embodiments 23-28, wherein the composition comprises a pharmaceutically acceptable carrier or excipient.
30. The vaccine composition of any one of embodiments 23-29, wherein the Mycobacterium tuberculosis microbe is inactivated by contacting it with UV light in the presence of riboflavin.
31. A method for treating and/or preventing a mycobacterial infection in a subject in need thereof, the method comprising administering to the subject an effective amount of the vaccine composition of any one of embodiments 18-30.
32. The method of embodiment 31, wherein the subject is a mammal.
33. The method of embodiment 32, wherein the subject is a human.
34. The method of any one of embodiments 31-33, wherein the vaccine is administered intramuscularly.
35. The method of any one of embodiments 31-33, wherein the vaccine is administered subcutaneously.
36. The method of any one of embodiments 31-35, wherein the method comprises administering a booster dose of the vaccine composition.
37. The method of embodiment 36, wherein the booster dose is administered about 1 week, about 2 weeks, about 3 weeks, about 4 weeks, about 2 months, about 3 months, about 4 months, about 5 months, about 6 months, or about 1 year after administering the vaccine composition.
38. A method for treating and/or preventing a mycobacterial infection in a subject in need thereof, the method comprising administering to the subject a first vaccine composition, comprising an effective amount of the vaccine composition of any one of embodiments 18-30; and a second vaccine composition, comprising an effective amount of the vaccine composition of any one of embodiments 18-30.
39. The method of embodiment 38, wherein the number of microbial cells in the first vaccine composition is greater than the number of microbial cells in the second vaccine composition.
40. The method of embodiment 38, wherein the number of microbial cells in the first vaccine composition is less than the number of microbial cells in the second vaccine composition.
41. The method of embodiment 38, wherein the number of microbial cells in the first vaccine composition is about the same as the number of microbial cells in the second vaccine composition.
42. The method of any one of embodiments 38-41, wherein the second vaccine composition is administered about 1 week, about 2 weeks, about 3 weeks, about 4 weeks, about 2 months, about 3 months, about 4 months, about 5 months, about 6 months, or about 1 year after the first vaccine composition.
43. The method of any one of embodiments 31-42, wherein the mycobacterial infection is tuberculosis.
44. A method for treating and/or preventing a mycobacterial infection in a subject in need thereof, the method comprising administering to the subject a first vaccine composition comprising an effective amount of attenuated, live Mycobacterium bovis, and a second vaccine composition, comprising an effective amount of the vaccine composition of any one of embodiments 18-30.
45. The method of embodiment 44, wherein the number of microbial cells in the first vaccine composition is greater than the number of microbial cells in the second vaccine composition.
46. The method of embodiment 44, wherein the number of microbial cells in the first vaccine composition is less than the number of microbial cells in the second vaccine composition.
47. The method of embodiment 44, wherein the number of microbial cells in the first vaccine composition is about the same as the number of microbial cells in the second vaccine composition.
48. The method of any one of embodiments 44-47, wherein the second vaccine composition is administered about 1 week, about 2 weeks, about 3 weeks, about 4 weeks, about 2 months, about 3 months, about 4 months, about 5 months, about 6 months, or about 1 year after the first vaccine composition.
49. The method of any one of embodiments 44-48, wherein the first vaccine composition is administered subcutaneously or intramuscularly.
50. The method of any one of embodiments 44-48, wherein the second vaccine composition is administered subcutaneously or intramuscularly.
51. The method of any one of embodiments 44-50, wherein the mycobacterial infection is tuberculosis.
52. The method of any one of embodiments 44-51, wherein the second vaccine composition comprises the BCG vaccine.
53. A method for producing a microbial vaccine, the method comprising (i) providing a plurality of microbes, and (ii) inactivating the microbes by contacting them with a dose of UV light in the presence of riboflavin, wherein the microbes are Mycobacterium tuberculosis microbes.
54. The method of embodiment 53, wherein the dose of UV light is about 100 Joules to about 25,000 Joules.
55. The method of embodiment 53, wherein the dose of UV light is about 17,000 Joules.
56. The method of any one of embodiments 53-55, wherein the method comprises purifying the inactivated microbes.
57. The method of any one of embodiments 53-55, wherein the microbes are Mycobacterium tuberculosis strain H37Rv.

1. A method for inactivating a microbe, the method comprising contacting the microbe with a dose of UV light in the presence of riboflavin, wherein the microbe belongs to the genus Mycobacterium.

2. The method of claim 1, wherein the microbe is capable of causing disease in a subject.

3. (canceled)

4. The method of claim 1, wherein the microbe belongs to the Mycobacterium tuberculosis complex.

5. The method of claim 1, wherein the microbe is selected from the group consisting of Mycobacterium tuberculosis, Mycobacterium africanum, Mycobacterium orygis, Mycobacterium bovis, Mycobacterium microti, Mycobacterium canetti, Mycobacterium caprae, Mycobacterium pinnipedii, Mycobacterium suricattae, and Mycobacterium mungi.

6. The method of claim 5, wherein the microbe is Mycobacterium tuberculosis.

7. The method of claim 6, wherein the microbe is Mycobacterium tuberculosis strain H37Rv.

8. The method of claim 1, wherein the microbe is resistant to an antibiotic.

9. (canceled)

10. The method of claims 1, wherein the dose of UV light is about 100 Joules to about 25,000 Joules.

11. The method of claim 1, wherein the dose of UV light is about 17,000 Joules.

12. The method of claim 1, wherein the method comprises altering the genome of the microbe.

13. The method of claim 12, wherein the method comprises selectively oxidizing one or more guanine bases in a nucleic acid of the microbe.

14. The method of claim 13, wherein the nucleic acid of the microbe is a DNA or an RNA.

15. The method of claim 14, wherein the method does not comprise substantially altering the structure of antigens on the surface of the microbe.

16-17. (canceled)

18. A vaccine composition, comprising a microbe inactivated according to claim 1.

19-30. (canceled)

31. A method for treating and/or preventing a mycobacterial infection in a subject in need thereof, the method comprising administering to the subject an effective amount of the vaccine composition of claim 18.

32-42. (canceled)

43. The method of claim 31, wherein the mycobacterial infection is tuberculosis.

44-51. (canceled)

53. A method for producing a microbial vaccine, the method comprising (i) providing a plurality of microbes, and (ii) inactivating the microbes by contacting them with a dose of UV light in the presence of riboflavin, wherein the microbes are Mycobacterium tuberculosis microbes. a

[symbol]

54. The method of claim 53, wherein the dose of UV light is about 100 Joules to about 25,000 Joules.

55. The method of claim 53, wherein the dose of UV light is about 17,000 Joules.

56. The method of claim 53, wherein the method comprises purifying the inactivated microbes.

57. (canceled)


SEQUENCE LISTING (2 sequences)

SEQ ID NO: 1 - DNA, 22 bp, Artificial Sequence
  Adjuvant oligonucleotide
tgactgtgaacgttcgagatga

SEQ ID NO: 2 - DNA, 20 bp, Artificial Sequence
  Adjuvant oligonucleotide
tccatgacgttcctgatgct